(19) United States
(12) Patent Application Publication
(10) Pub. No.: US 2026/0000266 A1
(43) Pub. Date: Jan. 1, 2026

(54) ENDOSCOPE SYSTEM, CONTROL DEVICE, AND CONTROL METHOD FOR ENDOSCOPE SYSTEM

(71) Applicant: OLYMPUS MEDICAL SYSTEMS CORP., Tokyo (JP)

(72) Inventors: Tatsuya HORIUCHI, Sagamihara-shi (JP); Ryota YANAGAWA, Hachioji-shi (JP); Kosuke KISHI, Mitaka-shi (JP)

(73) Assignee: OLYMPUS MEDICAL SYSTEMS CORP., Tokyo (JP)

(21) Appl. No.: 19/254,438

(22) Filed: Jun. 30, 2025

Related U.S. Application Data
(60) Provisional application No. 63/666,384, filed on Jul. 1, 2024.

(51) Int. Cl. A61B 1/00

(52) U.S. Cl. CPC A61B 1/00006; A61B 1/00039; A61B 1/00068; A61B 1/00119

(57) ABSTRACT

A control device for use with an endoscope is configured to: set a first pipe line to a closed state, a second pipe line to an open state, and a third pipe line to a closed state, to cause liquid to be ejected from an air and water supply portion; set the first pipe line to an open state, the second pipe line to a closed state, and the third pipe line to the closed state, to cause gas to be ejected from the air and water supply portion; and set the first pipe line to the closed state, the second pipe line to the open state, and the third pipe line to an open state, to reduce a pressure in a water supply tank.

### CROSS-REFERENCE TO RELATED APPLICATIONS

Priority is claimed on U.S. Provisional Application No. 63/666,384, filed on Jul. 1, 2024, the content of which is incorporated herein by reference.

### FIELD

The present disclosure relates to an endoscope system, a control device, and a control method for an endoscope system.

### BACKGROUND

Conventionally, endoscopes have been used for observing and treating the inside of hollow organs such as the digestive tract, or the like. An objective lens for observing an object to be observed is provided at a distal end of the endoscope.

In addition, in order to improve visibility through the objective lens, an air and water supply portion that ejects liquid such as water and gas such as air onto the objective lens is often provided at the distal end of the endoscope (for example, Japanese Patent No. 5963979). An endoscope equipped with an air and water supply portion cleans an objective lens by ejecting a liquid, and then removes any liquid remaining on the objective lens by ejecting a gas onto the objective lens. The liquid and the gas are sent to the air and water supply portion via a common pipe line.

### SUMMARY

When the liquid remains in the pipe line that sends the liquid and gas to the air and water supply portion, there is a possibility that the liquid will be ejected along with the gas when the gas is ejected from the air and water supply portion, which may hinder the process of removing the liquid on the objective lens.

The present disclosure provides an endoscope system, a control device, and a control method for an endoscope system that can curb a liquid being ejected along with a gas when the gas is ejected from an air and water supply portion capable of ejecting both the liquid and gas.

According to an aspect of the present disclosure, a control device for use with an endoscope is configured to: set a first pipe line to a closed state, a second pipe line to an open state, and a third pipe line to a closed state, to cause liquid to be ejected from an air and water supply portion; set the first pipe line to an open state, the second pipe line to a closed state, and the third pipe line to the closed state, to cause gas to be ejected from the air and water supply portion; and set the first pipe line to the closed state, the second pipe line to the open state, and the third pipe line to an open state, to reduce a pressure in a water supply tank.

According to an aspect of the present disclosure, an endoscope system includes: the above-described control device; an air and water supply portion; a supply pipe line configured to supply gas or liquid to the air and water supply portion; the first pipe line and the second pipe line which are connected to the supply pipe line; an air supply drive portion connected to the first pipe line and configured to supply gas; a water supply tank connected to the second pipe line; and the third pipe line branching off from the first pipe line and configured to open the first pipe line to an atmosphere.

According to an aspect of the present disclosure, a control device, which controls an endoscope system, performs: a water supply step of controlling a switching portion to set a first pipe line to a closed state, a second pipe line to an open state, and a third pipe line to a closed state, and driving an air supply drive portion to eject a liquid from an air and water supply portion; an air supply step of setting the first pipe line to an open state, the second pipe line to a closed state, and the third pipe line to a closed state, and driving the air supply drive portion to eject a gas from the air and water supply portion; and an atmosphere opening step of setting the first pipe line to the closed state, the second pipe line to the open state, and the third pipe line to the open state to reduce a pressure in a water supply tank.

According to the endoscope system, the control device, and the control method for an endoscope system of the present disclosure, it is possible to provide an endoscope system, a control device, and a control method for an endoscope system that can curb a liquid being ejected along with a gas when the gas is ejected from an air and water supply portion capable of ejecting both the liquid and gas.

### BRIEF DESCRIPTION OF THE DRAWINGS

FIG. 1 is an overall view of an endoscope system according to an embodiment.

FIG. 2 is a diagram showing an endoscope and an operation device of the endoscope system used by an operator.

FIG. 3 is a diagram showing an insertion portion of the endoscope.

FIG. 4 is a diagram showing the insertion portion of the endoscope.

FIG. 5 is a cross-sectional view showing a part of a bending portion of the insertion portion.

FIG. 6 is an enlarged view of a joint ring of the bending portion in a region E shown in FIG. 5.

FIG. 7 is a cross-sectional view of the bending portion taken along line C1-C1 in FIGS. 5 and 6.

FIG. 8 is a diagram showing a first attaching and detaching portion before it is mounted on a drive device of the endoscope system.

FIG. 9 is a diagram showing an upper-lower bending wire attaching and detaching portion before it is mounted on the drive device.

FIG. 10 is a view showing the upper-lower bending wire attaching and detaching portion mounted on the drive device.

FIG. 11 is a functional block diagram of the drive device.

FIG. 12 is a perspective view of the operation device of the endoscope system.

FIG. 13 is a perspective view of the operation device when seen from the back.

FIG. 14 is a functional block diagram of an image control device of the endoscope system.

FIG. 15 is a functional block diagram of an air and water supply control device of the endoscope system.

FIG. 16 is a cross-sectional view showing an example of a switching portion of the endoscope system.

FIG. 17 is a diagram showing a non-operational step of a control method for the endoscope system.

FIG. 18 is a diagram showing an air supply step of the control method of the endoscope system.

FIG. 19 is a diagram showing a water supply step of the control method of the endoscope system.

FIG. 20 is a diagram showing an atmosphere opening step of the control method of the endoscope system.

FIG. 21 is a flowchart showing the control method of the endoscope system.

### DETAILED DESCRIPTION

An endoscope system 1000 according to an embodiment of the present disclosure will be described with reference to the drawings.

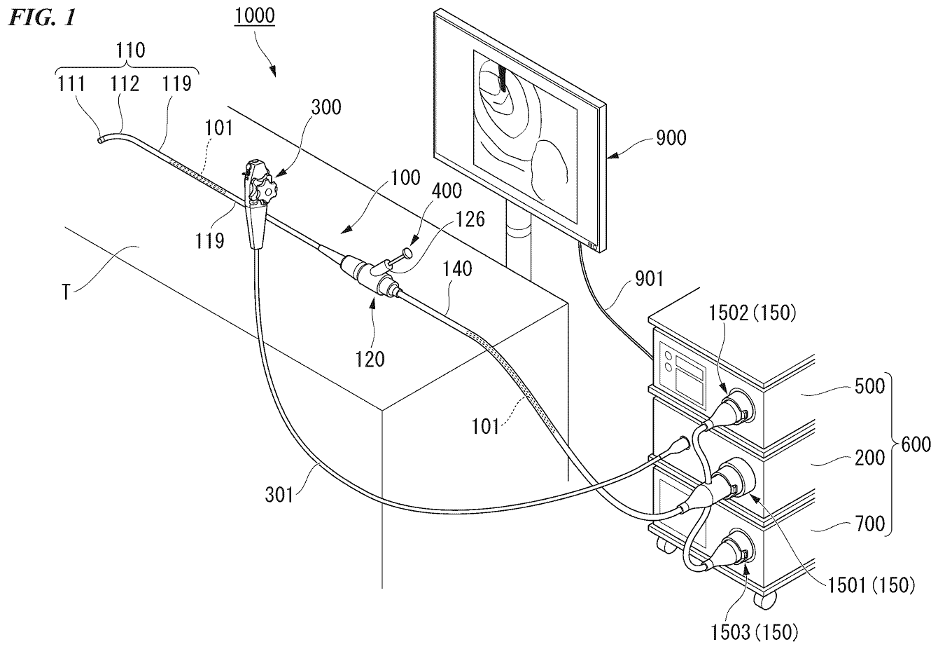

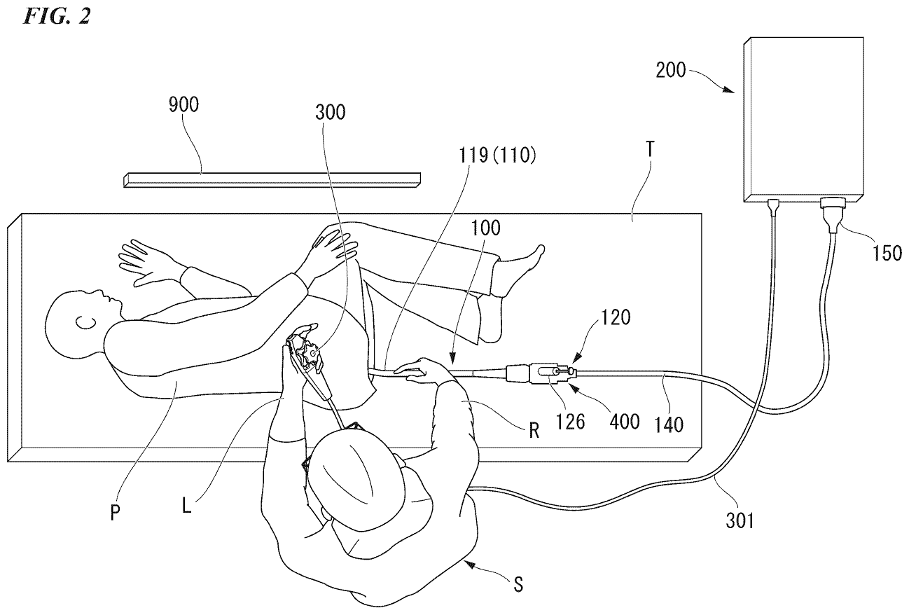

FIG. 1 is an overall view of the endoscope system 1000 according to this embodiment. FIG. 2 is a view showing an endoscope 100 and an operation device 300 of the endoscope system 1000 used by an operator S.

### [Endoscope System 1000]

The endoscope system 1000 is a medical system that observes and treats the inside of a body of a patient P lying on an operating table T. In this embodiment, the endoscope system 1000 is an electric endoscope system which is electrically driven.

The endoscope system 1000 includes an endoscope 100, a drive device 200, an air and water supply control device 700, an operation device 300, a treatment tool 400, an image control device 500, and a display device 900.

The endoscope 100 is a device that is inserted into a lumen of the patient P to observe and treat an affected area. The endoscope 100 is attachable to and detachable from a control device 600. An internal passage 101 is formed inside the endoscope 100.

The drive device 200 is detachably connected to the endoscope 100 and the operation device 300. The drive device 200 drives a built-in motor based on an operation input to the operation device 300 to electrically drive the endoscope 100.

The air and water supply control device 700 is detachably connected to the endoscope 100. The air and water supply control device 700 is also connected to the operation device 300 via the drive device 200. The air and water supply control device 700 drives a built-in pump or the like based on an operation input to the operation device 300 to perform air supply and water supply to the endoscope 100.

The operation device 300 is detachably connected to the drive device 200 via an operation cable 301. The operation device 300 may be capable of communicating with the drive device 200 by wireless communication rather than wired communication. The operator S can electrically drive the endoscope 100 by operating the operation device 300.

The treatment tool 400 is a device that is inserted through the internal passage 101 of the endoscope 100 into a lumen of the patient P to treat an affected area. In FIG. 1, the treatment tool 400 is inserted into the internal passage 101 of the endoscope 100 from a forceps port 126.

The image control device 500 is detachably connected to the endoscope 100 and acquires captured images from the endoscope 100. The image control device 500 causes the display device 900 to display the captured images acquired from the endoscope 100, as well as GUI images and CG images intended to provide information to the operator.

The drive device 200, the image control device 500, and the air and water supply control device 700 constitute the control device 600 that controls the endoscope system 1000. The control device 600 may further include peripheral devices such as a video printer. The drive device 200, the image control device 500, and the air and water supply control device 700 may be integrated into one device.

The display device 900 is a device capable of displaying images, such as an LCD, or the like. The display device 900 is connected to the image control device 500 via a display cable 901.

For example, the operator S operates the endoscope 100 inserted into the lumen from the anus of the patient P with his/her right hand R and operates the operation device 300 with his/her left hand L while observing the captured image displayed on the display device 900. Since the endoscope 100 and the operation device 300 are separate, the operator S can operate the endoscope 100 and the operation device 300 independently without them affecting each other.

### [Endoscope 100]

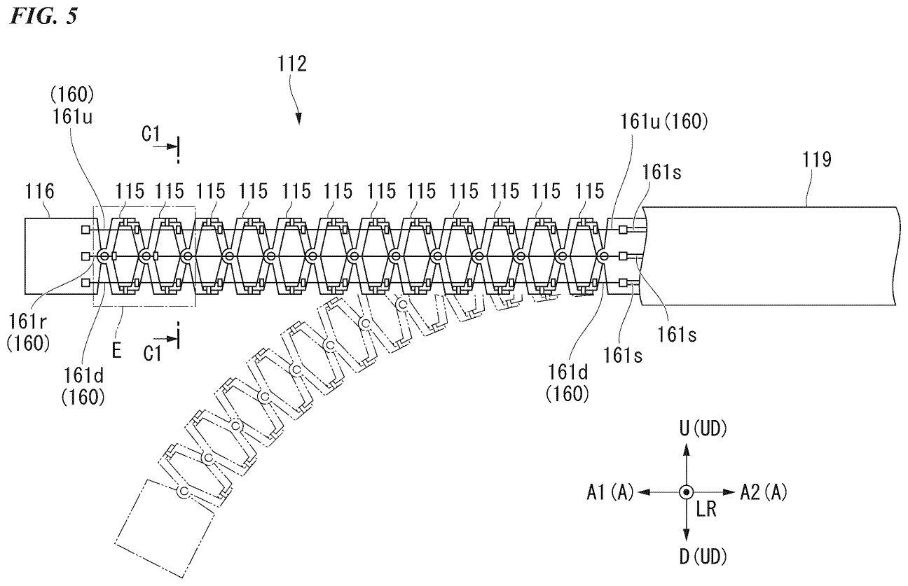

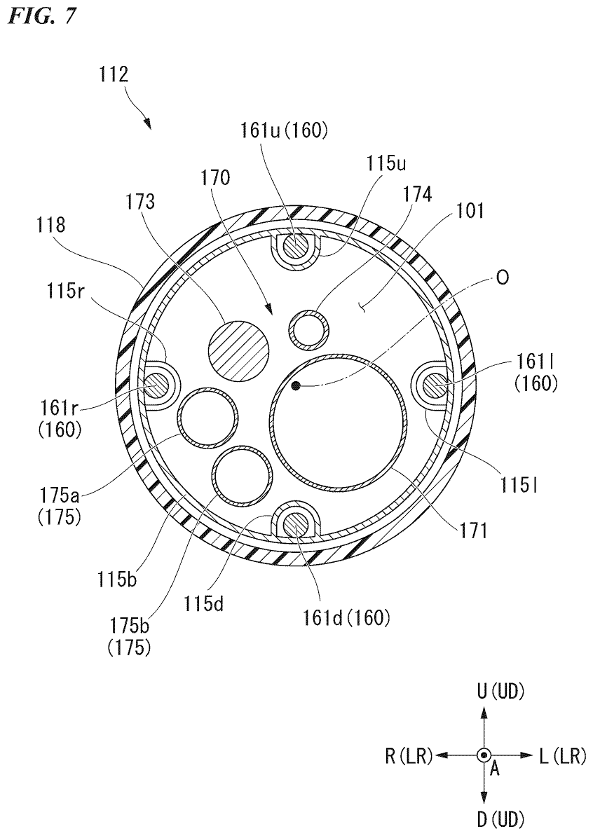

As shown in FIG. 1, the endoscope 100 includes an insertion portion 110, a connection portion 120, an extracorporeal flexible portion 140, an attaching and detaching portion 150, a bending wire 160 (refer to FIG. 5), and a built-in element 170 (refer to FIG. 7).

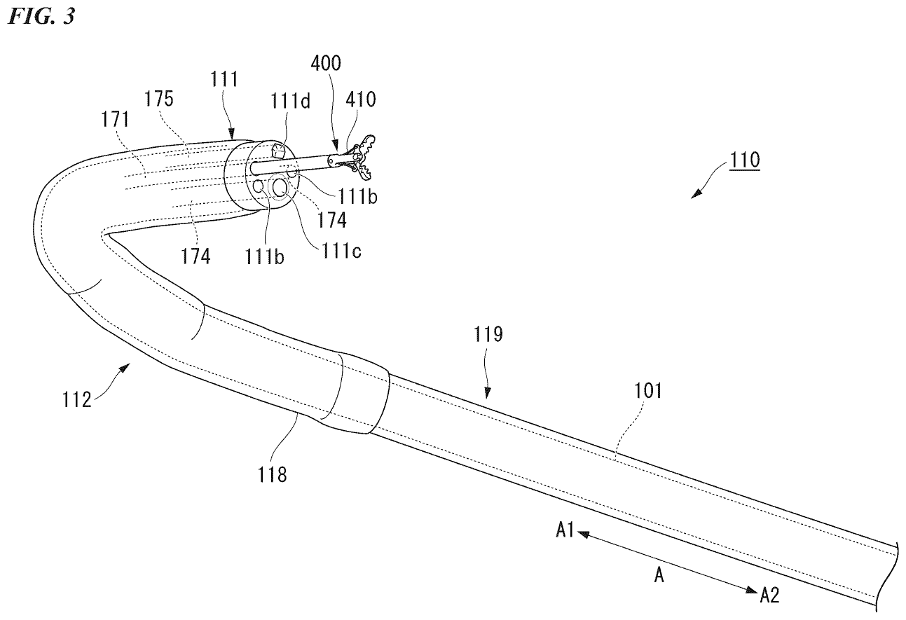

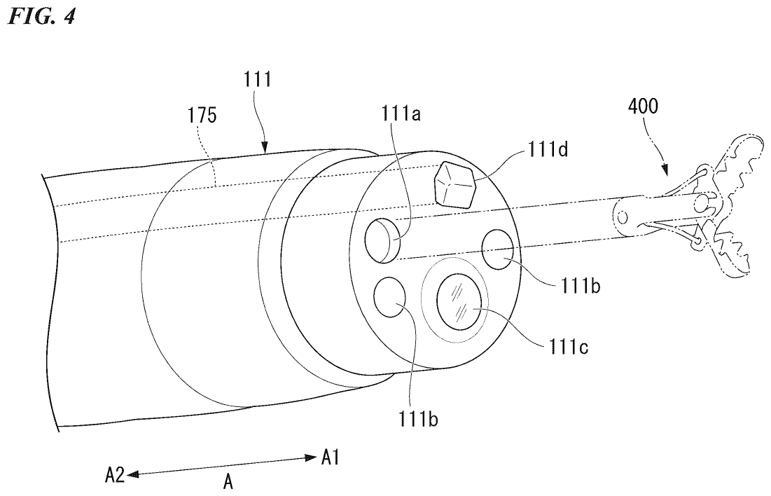

FIGS. 3 and 4 are diagrams showing the insertion portion 110 of the endoscope 100.

In the following description, as shown in FIGS. 3 and 4, in a longitudinal direction A of the endoscope 100, the side that is inserted into the lumen of the patient P is referred to as the “distal end side (distal side) A1,” and the side that is mounted in the drive device 200 is referred to as the “proximal end side (proximal side) A2.”

The insertion portion 110, the connection portion 120, the extracorporeal flexible portion 140, and the attaching and detaching portion 150 are connected in this order from the distal end side A1.

The internal passage 101 that extends in the longitudinal direction A of the endoscope 100 from a distal end of the insertion portion 110 to a proximal end of the attaching and detaching portion 150 is formed inside the endoscope 100. The bending wire 160 and the built-in element 170 are inserted into the internal passage 101.

The built-in element 170 includes a channel tube 171, an imaging cable 173, a light guide 174, and an air and water supply pipe line 175.

### [Insertion Portion 110]

The insertion portion 110 is an elongated member that is long and thin and can be inserted into a lumen. The insertion portion 110 has a distal end portion 111, a bending portion 112, and an intracorporeal flexible portion 119. The distal end portion 111, the bending portion 112, and the intracorporeal flexible portion 119 are connected in this order from the distal end side A1.

As shown in FIGS. 3 and 4, the distal end portion 111 has an opening portion 111a, an illumination portion 111b, an imaging portion 111c, and an air and water supply portion 111d.

The opening portion 111a is an opening that communicates with the channel tube 171. As shown in FIGS. 3 and 4, a treatment portion 410 such as a grasping forceps provided at a distal end of the treatment tool 400 that is inserted through the channel tube 171 protrudes and retracts from the opening portion 111a.

The illumination portion 111b is connected to the light guide 174 that guides illumination light, and emits illumination light that illuminates an imaging target.

The imaging portion 111c is an imaging device that includes an imaging element such as a CMOS, and captures an image of the imaging target. An imaging signal is sent to the image control device 500 via the imaging cable 173.

The air and water supply portion 111d is an air and water supply nozzle that has an opening facing the imaging portion 111c and can eject, to the imaging portion 111c, a gas or liquid flowing through the air and water supply pipe line 175. The air and water supply portion 111d ejects the gas or liquid in a direction perpendicular to the longitudinal direction A, for example.

FIG. 5 is a cross-sectional view of a part of the bending portion 112.

The bending portion 112 has a plurality of joint rings (also referred to as bending pieces) 115, a distal end portion 116 connected to distal ends of the plurality of joint rings 115, and an outer sheath 118 (refer to FIG. 3).

The plurality of joint rings 115 and the distal end portion 116 are connected in the longitudinal direction A inside the outer sheath 118. The shape and number of the joint rings 115 of the bending portion 112 are not limited to those shown in FIG. 5.

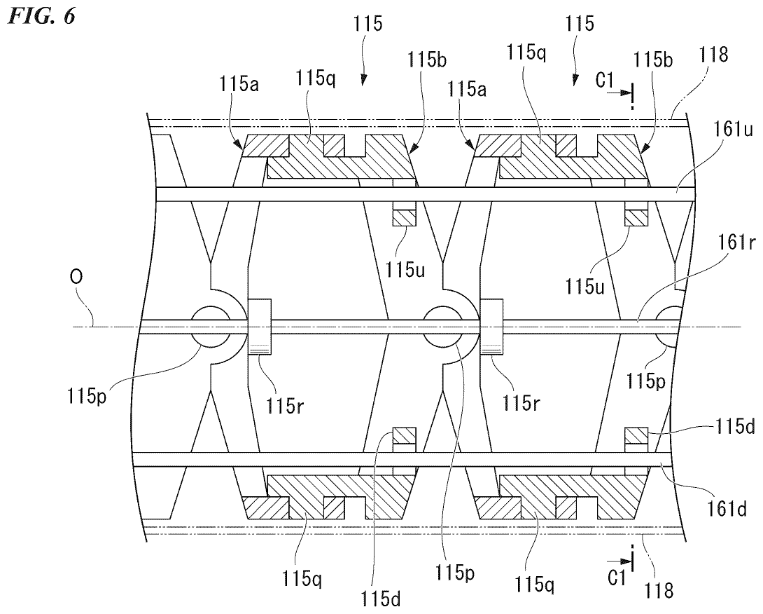

FIG. 6 is an enlarged view of the joint rings 115 in a region E shown in FIG. 5. The joint rings 115 are short tubular members made of a metal. The plurality of joint rings 115 are connected to each other so that internal spaces of the adjacent joint rings 115 are continuous.

Each of the joint rings 115 has a first joint ring 115a on the distal end side A1 and a second joint ring 115b on the proximal end side A2.

The first joint ring 115a and the second joint ring 115b are connected by a first pivot pin 115p so as to be rotatable in an up-down direction (also referred to as a “UD direction”) perpendicular to the longitudinal direction A.

Also, the first joint ring 115a and the second joint ring 115b are connected by a second pivot pin 115q so as to be rotatable in a left-right direction (also referred to as an “LR direction”) perpendicular to the longitudinal direction A and the UD direction.

The first joint rings 115a and the second joint rings 115b are alternately connected by the first pivot pins 115p and the second pivot pins 115q, and the bending portion 112 is freely bendable in a desired direction.

FIG. 7 is a cross-sectional view of the bending portion 112 taken along line C1-C1 in FIGS. 5 and 6.

An upper wire guide 115u and a lower wire guide 115d are formed on an inner circumferential surface of the second joint ring 115b.

The upper wire guide 115u and the lower wire guide 115d are disposed on both sides in the UD direction with a central axis O in the longitudinal direction A interposed therebetween.

A left wire guide 1151 and a right wire guide 115r are formed on the inner circumferential surface of the first joint ring 115a.

The left wire guide 1151 and the right wire guide 115r are disposed on both sides in the LR direction with the central axis O in the longitudinal direction A interposed therebetween.

The upper wire guide 115u, the lower wire guide 115d, the left wire guide 1151, and the right wire guide 115r have through holes formed in the longitudinal direction A, through which the bending wire 160 is inserted.

The bending wire 160 is a wire that bends the bending portion 112. The bending wire 160 passes through the internal passage 101 and extends to the attaching and detaching portion 150. As shown in FIG. 5 and FIG. 7, the bending wire 160 has an upper bending wire 161u, a lower bending wire 161d, a left bending wire 1611, a right bending wire 161r, and four wire sheaths 161s.

As shown in FIG. 5, each of the upper bending wire 161u, the lower bending wire 161d, the left bending wire 1611, and the right bending wire 161r is inserted through a wire sheath 161s.

A distal end of the wire sheath 161s is mounted on the joint ring 115 at the proximal end of the bending portion 112. The wire sheath 161s extends to the attaching and detaching portion 150.

The upper bending wire 161u and the lower bending wire 161d are wires that bend the bending portion 112 in the UD direction. The upper bending wire 161u is inserted through the upper wire guide 115u, and the lower bending wire 161d is inserted through the lower wire guide 115d.

Distal ends of the upper bending wire 161u and the lower bending wire 161d are fixed to the distal end portion 116 at the distal end of the bending portion 112, as shown in FIG. 5. The distal ends of the upper bending wire 161u and the lower bending wire 161d fixed to the distal end portion 116 are disposed on both sides in the UD direction with the central axis O in the longitudinal direction A interposed therebetween.

The left bending wire 1611 and the right bending wire 161r are wires that bend the bending portion 112 in the LR direction. The left bending wire 1611 is inserted through the left wire guide 1151. The right bending wire 161r is inserted through the right wire guide 115r.

Distal ends of the left bending wire 1611 and the right bending wire 161r are fixed to the distal end portion 116 of the bending portion 112 as shown in FIG. 5. The distal ends of the left bending wire 1611 and the right bending wire 161r fixed to the distal end portion 116 are disposed on both sides in the LR direction with the central axis O in the longitudinal direction A interposed therebetween.

The bending portion 112 can be bent freely in a desired direction by pulling or relaxing each of the bending wires 160 (the upper bending wire 161u, the lower bending wire 161d, the left bending wire 1611, and the right bending wire 161r).

As shown in FIG. 7, the bending wire 160, the channel tube 171, the imaging cable 173, the light guide 174, and the air and water supply pipe line 175 are inserted through the internal passage 101 formed inside the bending portion 112.

The intracorporeal flexible portion 119 is a long and flexible tubular member. The bending wire 160, the channel tube 171, the imaging cable 173, the light guide 174, and the air and water supply pipe line 175 are inserted through the internal passage 101 formed in the intracorporeal flexible portion 119.

As shown in FIG. 7, the air and water supply pipe line 175 includes an air supply pipe line 175a and a water supply pipe line 175b.

The air and water supply pipe line 175 is formed of a single pipe line at the distal end portion 111 and is connected to the air and water supply portion 111d. Furthermore, the air and water supply pipe line 175 branches into the air supply pipe line 175a and the water supply pipe line 175b at the bending portion 112.

The air supply pipe line 175a and the water supply pipe line 175b branching off from the air and water supply pipe line 175 are inserted through the internal passage 101 and extend to the attaching and detaching portion 150.

### [Connection Portion 120]

The connection portion 120 is a member that connects the intracorporeal flexible portion 119 and the extracorporeal flexible portion 140 of the insertion portion 110, as shown in FIG. 1. The connection portion 120 has the forceps port 126 which is an insertion port for inserting the treatment tool 400.

### [Extracorporeal Flexible Portion 140]

The extracorporeal flexible portion 140 is a long tubular member. The bending wire 160, the imaging cable 173, the light guide 174, and the air and water supply pipe line 175 (the air supply pipe line 175a and the water supply pipe line 175b) are inserted through the internal passage 101 formed inside the extracorporeal flexible portion 140.

### [Attaching and Detaching Portion 150]

As shown in FIG. 1, the attaching and detaching portion 150 includes a first attaching and detaching portion 1501 that is mounted on the drive device 200, a second attaching and detaching portion 1502 that is mounted on the image control device 500, and a third attaching and detaching portion 1503 that is mounted on the air and water supply control device 700. The first attaching and detaching portion 1501, the second attaching and detaching portion 1502 and the third attaching and detaching portion 1503 may be an integrated attaching and detaching portion.

The internal passage 101 formed inside the extracorporeal flexible portion 140 branches into the first attaching and detaching portion 1501, the second attaching and detaching portion 1502, and the third attaching and detaching portion 1503.

The bending wire 160 is inserted through the first attaching and detaching portion 1501. The imaging cable 173 and the light guide 174 are inserted through the second attaching and detaching portion 1502. The air supply pipe line 175a and the water supply pipe line 175b are inserted through the third attaching and detaching portion 1503.

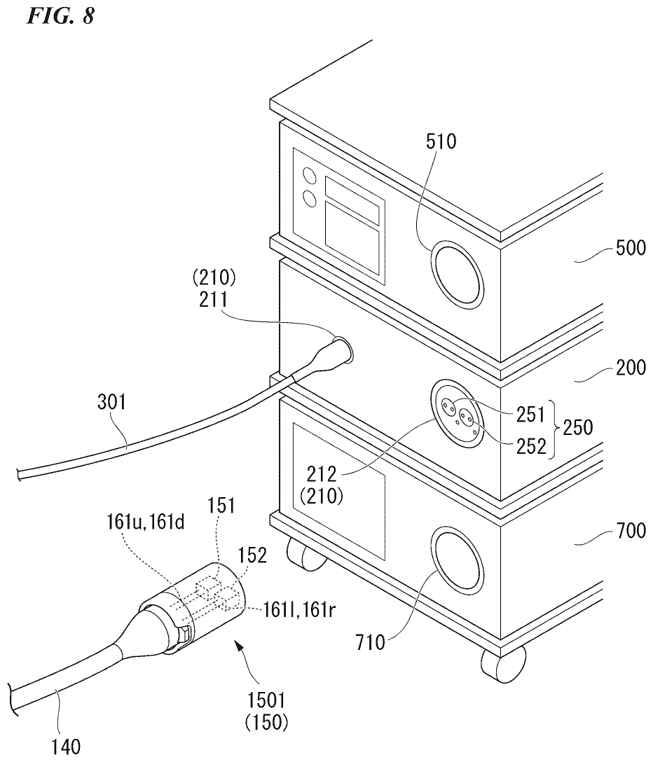

FIG. 8 is a diagram showing the first attaching and detaching portion 1501 before it is mounted on the drive device 200.

The first attaching and detaching portion 1501 has an upper-lower bending wire attaching and detaching portion 151 and a left-right bending wire attaching and detaching portion 152.

The upper-lower bending wire attaching and detaching portion 151 is a mechanism that detachably connects the wires (the upper bending wire 161u and the lower bending wire 161d) that bend the bending portion 112 in the UD direction to the drive device 200.

The left-right bending wire attaching and detaching portion 152 is a mechanism that detachably connects the wires (the left bending wire 1611 and the right bending wire 161r) that bend the bending portion 112 in the LR direction to the drive device 200.

The left-right bending wire attaching and detaching portion 152 has the same structure as the upper-lower bending wire attaching and detaching portion 151, and thus will not be illustrated or described.

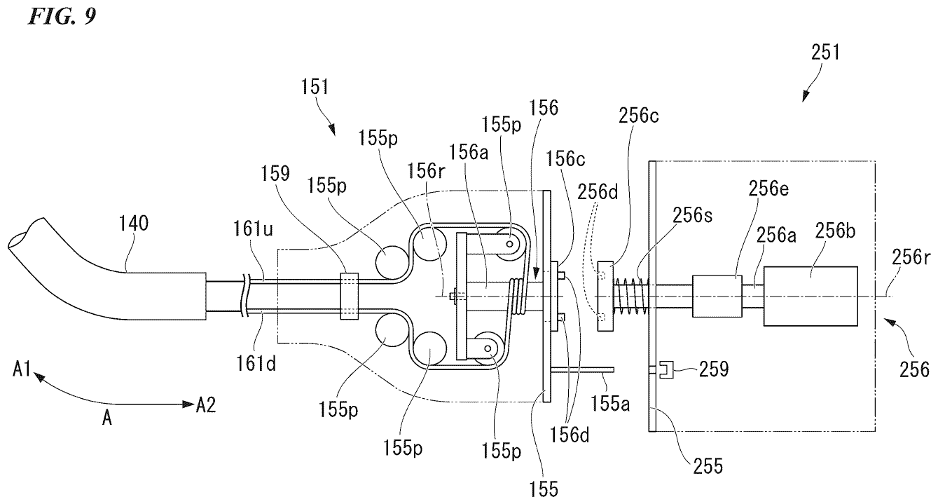

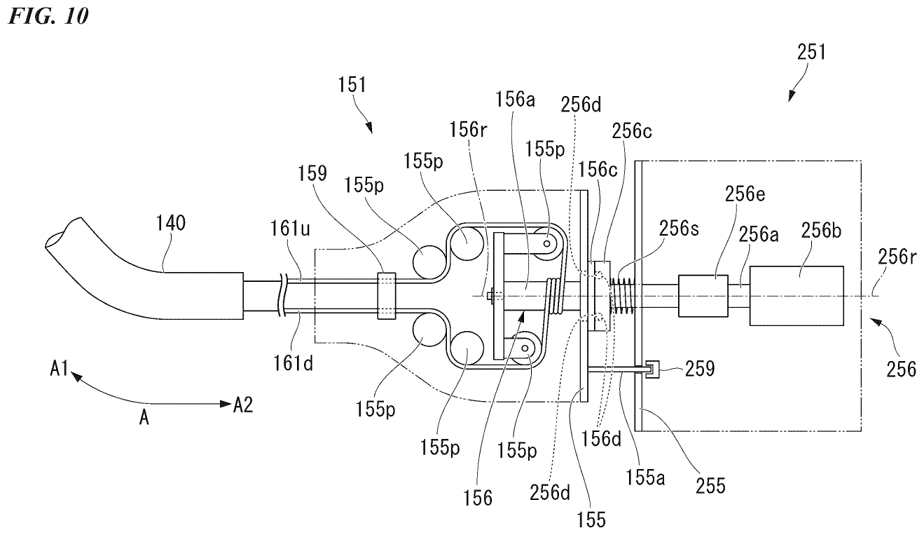

FIG. 9 is a diagram showing the upper-lower bending wire attaching and detaching portion 151 before it is mounted on the drive device 200. FIG. 10 is a diagram showing the upper-lower bending wire attaching and detaching portion 151 mounted on the drive device 200.

The upper-lower bending wire attaching and detaching portion 151 includes a support member 155, a rotating drum 156, and a tension sensor 159.

The support member 155 supports the rotating drum 156. The support member 155 includes an attachment and detachment detection dog 155a exposed on the proximal end side A2 of the upper-lower bending wire attaching and detaching portion 151, and a plurality of bend pulleys 155p.

The bend pulley 155p changes a transport direction of the upper bending wire 161u that is inserted through the extracorporeal flexible portion 140, and guides the upper bending wire 161u to the rotating drum 156. In addition, the bend pulley 155p changes the transport direction of the lower bending wire 161d that is inserted through the extracorporeal flexible portion 140, and guides the lower bending wire 161d to the rotating drum 156.

The rotating drum 156 is supported by the support member 155 so as to be rotatable about a drum rotation axis 156r extending in the longitudinal direction A. The rotating drum 156 includes a winding pulley 156a and a coupling portion 156c.

The winding pulley 156a pulls or sends the upper bending wire 161u and the lower bending wire 161d by rotating about the drum rotation axis 156r. When seen from the distal end side A1 toward the proximal end side A2, the winding pulley 156a rotates clockwise, whereby the upper bending wire 161u is wound around the winding pulley 156a and pulled, and the lower bending wire 161d is sent out of the winding pulley 156a.

On the other hand, when the winding pulley 156a rotates counterclockwise, the upper bending wire 161u is sent out of the winding pulley 156a, and the lower bending wire 161d is wound around the winding pulley 156a and pulled. With this configuration, even when the upper bending wire 161u and the lower bending wire 161d are advanced and retreated by a large amount, the pulled portions are stored compactly and do not take up space.

The upper bending wire 161u and the lower bending wire 161d have a larger diameter at the portion wound around the winding pulley 156a than at the other portions. Therefore, the upper bending wire 161u and the lower bending wire 161d can be suitably prevented from being pinched between the winding pulley 156a and the support member 155. In addition, stretching of the upper bending wire 161u and the lower bending wire 161d due to pulling or relaxing can be suitably prevented.

The upper bending wire 161u and the lower bending wire 161d may have a larger diameter at the portion passing through the extracorporeal flexible portion 140 than at the portion passing through the insertion portion 110. Thus, the insertion portion 110 that is inserted into the body can be made thinner. Furthermore, by making the diameter of the portion of the wire that passes outside the body thicker, the stretching of the upper bending wire 161u and the lower bending wire 161d is curbed, and controllability of a bending operation for the bending portion 112 is improved.

The coupling portion 156c is a disk member that rotates about the drum rotation axis 156r. The coupling portion 156c is fixed to a proximal end of the winding pulley 156a and rotates integrally with the winding pulley 156a. The coupling portion 156c is exposed on the proximal end side A2 of the upper-lower bending wire attaching and detaching portion 151. Two fitting protrusion portions 156d are formed on a surface of the coupling portion 156c on the proximal end side A2. The two fitting protrusion portions 156d are formed on both sides with the drum rotation axis 156r interposed therebetween.

The tension sensor 159 detects a tension of each of the upper bending wire 161u and the lower bending wire 161d. A detection result of the tension sensor 159 is acquired by a drive controller 260.

### [Drive Device 200]

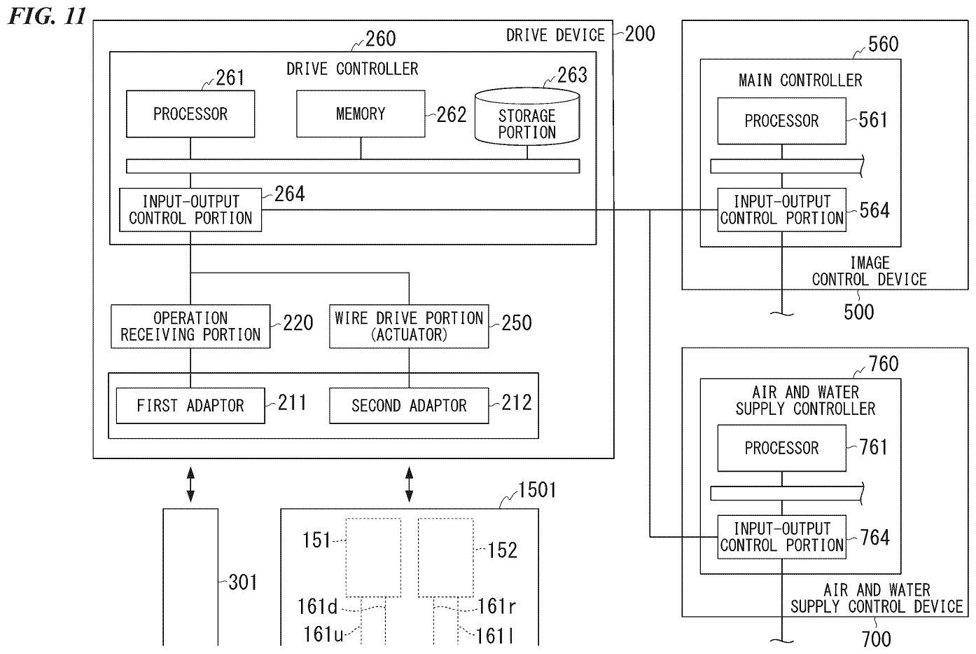

FIG. 11 is a functional block diagram of the drive device 200.

The drive device 200 includes a drive adaptor 210, an operation receiving portion 220, a wire drive portion (an actuator) 250, and the drive controller 260.

The drive adaptor 210 includes a first adaptor 211 and a second adaptor 212.

The first adaptor 211 is an adaptor to which the operation cable 301 is detachably connected.

The second adaptor 212 is an adaptor to which the first attaching and detaching portion 1501 of the endoscope 100 is detachably connected.

The operation receiving portion 220 receives an operation input from the operation device 300 via the operation cable 301. When the operation device 300 and the drive device 200 communicate with each other by wireless communication rather than wired communication, the operation receiving portion 220 has a known wireless reception module.

The wire drive portion (the actuator) 250 is coupled with the upper-lower bending wire attaching and detaching portion 151 and the left-right bending wire attaching and detaching portion 152 to drive the bending wire 160.

As shown in FIG. 8, the wire drive portion 250 has an upper-lower bending wire drive portion (a first actuator) 251 and a left-right bending wire drive portion (a second actuator) 252.

The upper-lower bending wire drive portion 251 is a mechanism that is coupled with the upper-lower bending wire attaching and detaching portion 151 and drives the wires (the upper bending wire 161u and the lower bending wire 161d) that bend the bending portion 112 in the UD direction.

The left-right bending wire drive portion 252 is a mechanism that is coupled with the left-right bending wire attaching and detaching portion 152 and drives the wires (the left bending wire 1611 and the right bending wire 161r) that bend the bending portion 112 in the LR directions.

The left-right bending wire drive portion 252 has the same structure as the upper-lower bending wire drive portion 251, and thus will not be illustrated or described.

As shown in FIGS. 9 and 10, the upper-lower bending wire drive portion 251 includes a support member 255, a bending wire drive portion 256, and an attachment and detachment sensor 259.

The bending wire drive portion 256 is coupled with the rotating drum 156 of the upper-lower bending wire attaching and detaching portion 151 to drive the upper bending wire 161u and the lower bending wire 161d. The bending wire drive portion 256 includes a shaft 256a, a motor portion 256b, a coupled portion 256c, a torque sensor 256e, and an elastic member 256s.

The shaft 256a is supported by the support member 255 so as to be rotatable about a shaft rotation axis 256r and movable back and forth in the longitudinal direction A. When the first attaching and detaching portion 1501 of the endoscope 100 is mounted on the drive device 200, the shaft rotation axis 256r coincides with the drum rotation axis 156r.

The motor portion 256b includes a motor such as a DC motor, a motor driver that drives the motor, and a motor encoder. The motor rotates the shaft 256a about the shaft rotation axis 256r. The motor driver is controlled by the drive controller 260.

The coupled portion 256c is a disk member that rotates about the shaft rotation axis 256r. The coupled portion 256c is fixed to a distal end of the shaft 256a and rotates integrally with the shaft 256a.

As shown in FIG. 9, the coupled portion 256c is exposed on the distal end side A1 of the upper-lower bending wire drive portion 251. Two fitting recessed portions 256d are formed on a surface of the coupled portion 256c on the distal end side A1. The two fitting recessed portions 256d are formed on both sides with the shaft rotation axis 256r interposed therebetween.

As shown in FIG. 10, the fitting protrusion portion 156d and the fitting recessed portion 256d are fitted to each other, and the coupling portion 156c and the coupled portion 256c are coupled with each other. As a result, the rotation of the shaft 256a by the motor portion 256b is transmitted to the rotating drum 156.

When seen from the distal end side A1 toward the proximal end side A2, the shaft 256a rotates clockwise, whereby the upper bending wire 161u is pulled, and the lower bending wire 161d is sent out. Conversely, the shaft 256a rotates counterclockwise, whereby the upper bending wire 161u is sent out, and the lower bending wire 161d is pulled.

The torque sensor 256e detects rotation torque of the shaft 256a about the shaft rotation axis 256r. A detection result of the torque sensor 256e is acquired by the drive controller 260.

The elastic member 256s is, for example, a compression spring, with a distal end portion in contact with the coupled portion 256c and a proximal end portion in contact with the support member 255. The elastic member 256s biases the coupled portion 256c toward the distal end side A1. As shown in FIG. 10, when the coupling portion 156c is mounted, the coupled portion 256c moves to the proximal end side A2 together with the shaft 256a.

As shown in FIG. 10, the attachment and detachment sensor 259 detects attachment and detachment of the upper-lower bending wire attaching and detaching portion 151 to/from the upper-lower bending wire drive portion 251 by detecting engagement and disengagement with the attachment and detachment detection dog 155a. A detection result of the attachment and detachment sensor 259 is acquired by the drive controller 260.

The drive controller 260 controls the entire drive device 200. The drive controller 260 acquires the operation input received by the operation receiving portion 220. The drive controller 260 controls the wire drive portion 250 on the basis of the acquired operation input.

The drive controller 260 is a computer capable of executing programs and including a processor 261, a memory 262, a storage portion 263 capable of storing programs and data, and an input-output control portion 264.

Functions of the drive controller 260 are realized by the processor executing a program. At least some of the functions of the drive controller 260 may be realized by a dedicated logic circuit.

The drive controller 260 desirably has high calculation performance in order to control a plurality of motors that drive a plurality of bending wires 160 with high precision.

The drive controller 260 may further include components other than the processor 261, the memory 262, the storage portion 263, and the input-output control portion 264.

### [Operation Device 300]

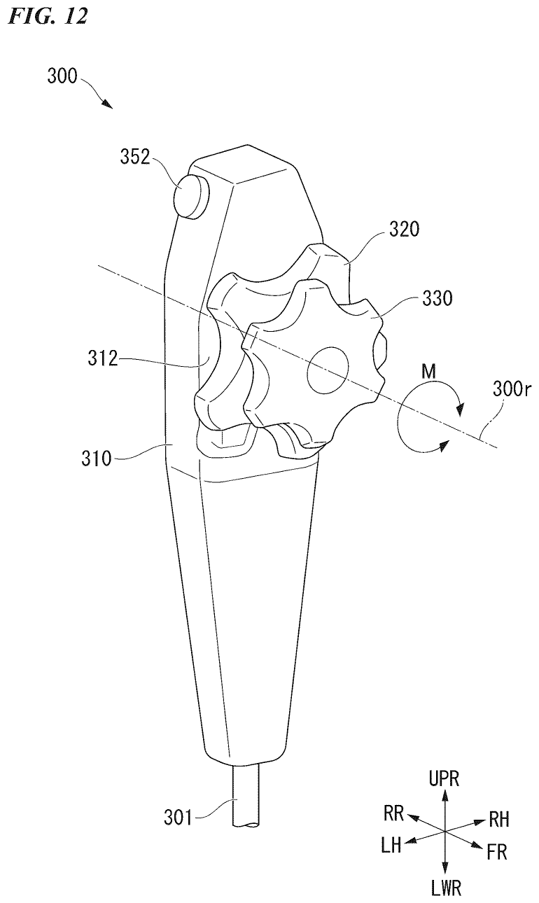

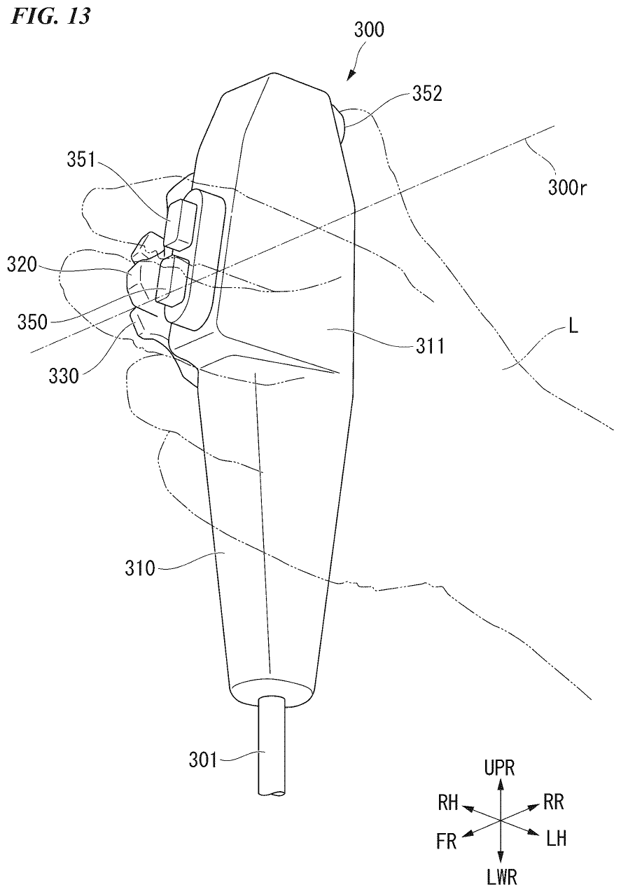

FIG. 12 is a perspective view of the operation device 300. FIG. 13 is a perspective view of the operation device 300 when seen from a back surface 311.

The operation device 300 is a device to which an operation for driving the endoscope 100 is input. The input operation input is transmitted to the drive device 200 via the operation cable 301.

The operation device 300 includes an operation portion main body 310, a first angle knob 320, a second angle knob 330, a first button 350, a second button 351, and a third button 352.

The operation portion main body 310 is formed in a substantially cylindrical shape that can be held by a left hand L of the operator S. As shown in FIG. 13, the operation portion main body 310 is formed with a back surface 311 that can follow a palm of the left hand L of the operator S. The operation cable 301 is connected to an end portion of the operation portion main body 310 in the longitudinal direction.

The first angle knob 320 and the second angle knob 330 are input elements to which a bending operation for bending the bending portion 112 is input.

The first angle knob 320 and the second angle knob 330 are rotatably mounted on the operation portion main body 310. The first angle knob 320 and the second angle knob 330 are mounted on a front surface 312 opposite to the back surface 311. The first angle knob 320 and the second angle knob 330 rotate in a rotation direction M about the same rotation axis 300r.

The first angle knob 320 and the second angle knob 330 have an encoder (not shown) that detects a rotation angle, the number of rotations, and the like in the rotation operation input to the first angle knob 320 and the second angle knob 330. A detection result of the encoder is transmitted to the drive device 200.

The drive controller 260 of the drive device 200 acquires the operation input transmitted by the operation device 300 and controls the wire drive portion 250.

The drive controller 260 controls the upper-lower bending wire drive portion 251 on the basis of the rotation operation input to the first angle knob 320, and drives the wires (the upper bending wire 161u and the lower bending wire 161d) that bend the bending portion 112 in the UD direction.

The drive controller 260 controls the left-right bending wire drive portion 252 on the basis of the rotation operation input to the second angle knob 330, and drives the wires (the left bending wire 1611 and the right bending wire 161r) that bend the bending portion 112 in the LR directions.

In the following description, a direction of the rotation axis 300r of the first angle knob 320 and the second angle knob 330 is defined as a “front-rear direction,” a direction in which the first angle knob 320 and the second angle knob 330 are mounted on the operation portion main body 310 is defined as a “forward direction FR,” and a direction opposite thereto is defined as a “rearward direction RR.” Further, the longitudinal direction of the operation portion main body 310 is defined as an “up-down direction,” the direction in which the operation cable 301 is mounted in the operation portion main body 310 is defined as “downward LWR,” and a direction opposite thereto is defined as “upward UPR.” In addition, a right direction facing the rear RR is defined as the “right side RH,” a direction opposite thereto is defined as the “left side LH,” and a direction toward the right side RH or the left side LH is defined as a “left-right direction.”

In this embodiment, the direction (the front-rear direction) in which the rotation axis 300r of the first angle knob 320 and the second angle knob 330 extend is a direction substantially perpendicular to the back surface 311 of the operation portion main body 310.

The first button 350, the second button 351, and the third button 352 are mounted upward UPR of the operation portion main body 310. The first button 350 and the second button 351 are operated by an index finger and a middle finger of the left hand L as shown in FIG. 13. The third button 352 is operated by a thumb of the left hand L as shown in FIG. 13.

Any function can be assigned to the first button 350, the second button 351, and the third button 352.

In this embodiment, the first button 350 functions as a detection portion capable of detecting an air supply operation for ejecting a gas such as air from the air and water supply portion 111d, and a water supply operation for ejecting a liquid such as water from the air and water supply portion 111d.

The first button (the detection portion) 350 can detect a first operation and a second operation that is different from the first operation. The first operation and the second operation are predetermined operations input by the operator S to the first button 350.

The first operation is, for example, a touch operation input by the index finger or middle finger of the left hand L of the operator S touching the first button 350.

In this case, the first button 350 detects the first operation when the operator S touches the first button 350 but does not press the first button 350.

The second operation is, for example, a push operation (or a switch operation) in which the first button 350 is pressed by the index finger or middle finger of the left hand L of the operator S.

In this case, when the operator S touches the first button 350 and presses the first button 350, the first button 350 detects the second operation.

The first button 350 may detect the first operation and the second operation by a two-stage pressing operation.

For example, the first operation may be an operation of pressing the first button 350 one step (a first push operation), and the second operation may be an operation of pressing the first button 350 further than the first push operation (a second push operation). The first push operation and the second push operation are operations in which the first button 350 is pressed by different amounts.

In this case, the operator S can input the first operation by lightly pressing the first button 350 and then can input the second operation by further pressing the first button 350.

Similarly to the first button 350, the second button 351 and the third button 352 may be configured to be able to detect the first operation and the second operation.

### [Image Control Device 500]

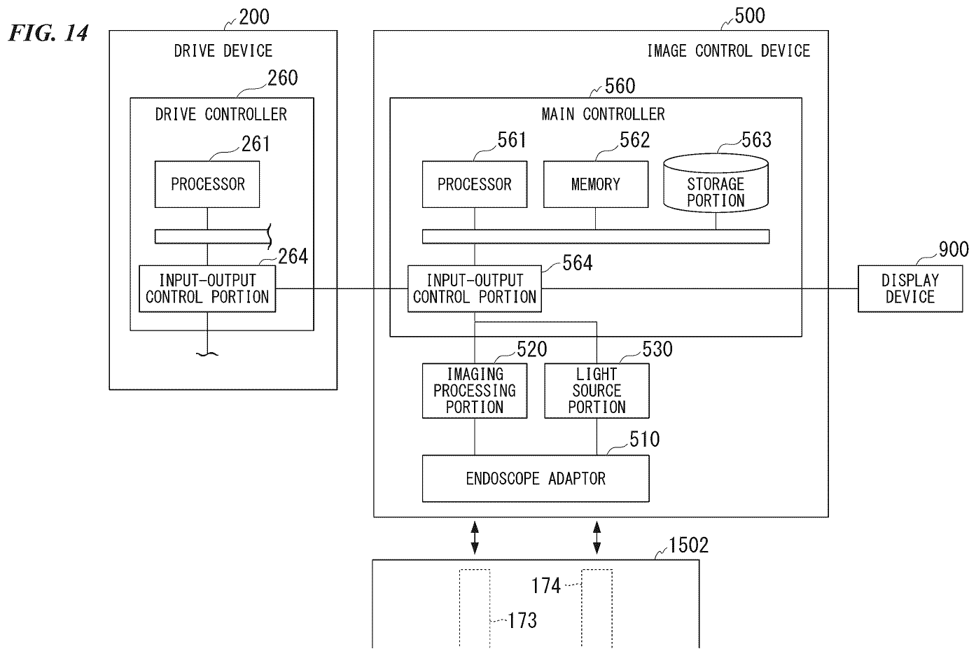

FIG. 14 is a functional block diagram of the image control device 500.

The image control device 500 controls the endoscope system 1000. The image control device 500 includes an endoscope adaptor 510, an imaging processing portion 520, a light source portion 530, and a main controller 560.

The endoscope adaptor 510 is an adaptor to which the second attaching and detaching portion 1502 of the endoscope 100 is detachably connected.

The imaging processing portion 520 converts an imaging signal acquired from the imaging portion 111c of the distal end portion 111 via the imaging cable 173 into a captured image.

The light source portion 530 generates illumination light to be radiated onto an imaging target. The illumination light generated by the light source portion 530 is guided to the illumination portion 111b of the distal end portion 111 via the light guide 174.

The main controller 560 is a computer capable of executing programs and including a processor 561, a memory 562, a storage portion 563 capable of storing programs and data, and an input-output control portion 564.

Functions of the main controller 560 are realized by the processor 561 executing a program. At least some of the functions of the main controller 560 may be realized by a dedicated logic circuit.

The main controller 560 includes the processor 561, the memory 562 from which a program can be read, the storage portion 563, and the input-output control portion 564.

The storage portion 563 is a non-volatile recording medium that stores the above-described programs and necessary data. The storage portion 563 is configured of, for example, a ROM, a hard disk, or the like. The program recorded in the storage portion 563 is read into the memory 562 and executed by the processor 561.

The input-output control portion 564 is connected to the imaging processing portion 520, the light source portion 530, the drive device 200, the display device 900, an input device (not shown), and a network device (not shown). The input-output control portion 564 transmits and receives data and control signals to and from connected devices based on the control of the processor 561.

The main controller 560 can perform image processing on the captured images acquired by the imaging processing portion 520. The main controller 560 can generate GUI images and CG images for the purpose of providing information to the operator S. The main controller 560 can display the captured images, the GUI images, and the CG images on the display device 900.

The main controller 560 is not limited to an integrated hardware device. For example, the main controller 560 may be configured by separating a part thereof into a separate hardware device and connecting the separated hardware device via a communication line. For example, the main controller 560 may be a cloud system in which the separated storage portion 563 is connected via a communication line.

The main controller 560 may further include components other than the processor 561, the memory 562, the storage portion 563, and the input-output control portion 564 shown in FIG. 14. For example, the main controller 560 may further include an image calculation portion that performs part or the whole of image processing and image recognition processing that were previously performed by the processor 561. By further including the image calculation portion, the main controller 560 can perform specific image processing and image recognition processing at high speed. The image calculation portion may be mounted in a separate hardware device connected via a communication line.

### [Air and Water Supply Control Device 700]

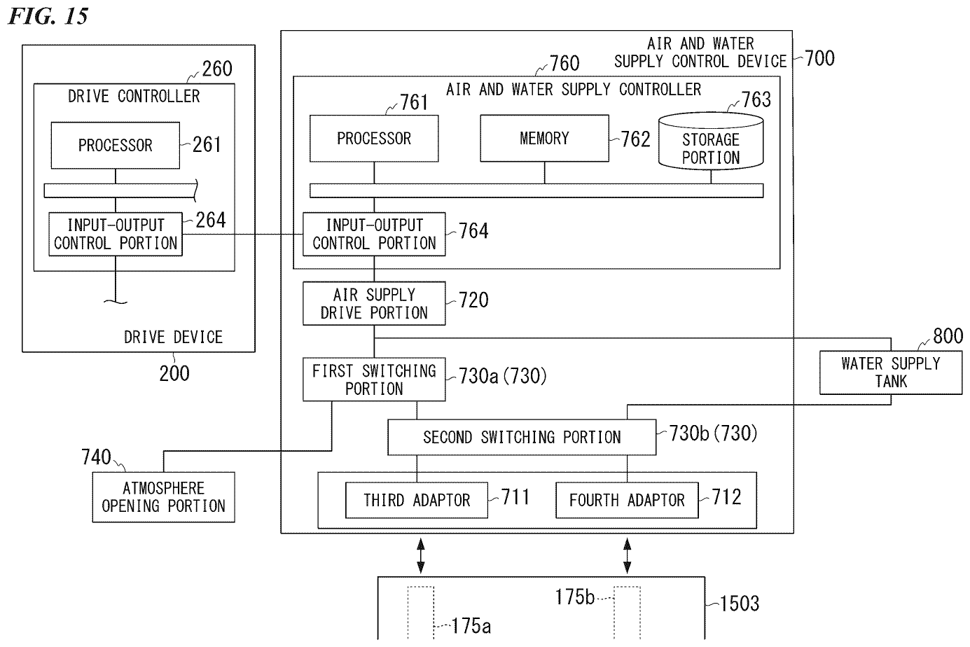

FIG. 15 is a functional block diagram of the air and water supply control device 700.

The air and water supply control device 700 controls the endoscope system 1000. The air and water supply control device 700 includes an air and water supply adaptor 710 (refer to FIG. 8), an air and water supply controller 760, an air supply drive portion 720, an atmosphere opening portion 740, and a switching portion 730.

The air and water supply adaptor 710 is an adaptor to which the third attaching and detaching portion 1503 of the endoscope 100 is detachably connected. The air and water supply adaptor 710 includes a third adaptor 711 and a fourth adaptor 712.

The third adaptor 711 is connected to the air supply pipe line 175a of the endoscope 100.

The fourth adaptor 712 is connected to the water supply pipe line 175b of the endoscope 100.

The air and water supply controller 760 is a computer capable of executing programs and including a processor 761, a memory 762, a storage portion 763 capable of storing programs and data, and an input-output control portion 764.

Functions of the air and water supply controller 760 are realized by the processor 761 executing a program. At least some of the functions of the air and water supply controller 760 may be realized by a dedicated logic circuit.

The air and water supply controller 760 has the processor 761, the memory 762 from which a program can be read, the storage portion 763, and the input-output control portion 764.

The storage portion 763 is a non-volatile recording medium that stores the above-described programs and necessary data. The storage portion 763 is configured of, for example, a ROM, a hard disk, or the like. The program recorded in the storage portion 763 is read into the memory 762 and executed by the processor 761.

The input-output control portion 764 is connected to the air supply drive portion 720, the switching portion 730, and the drive device 200. The input-output control portion 764 transmits and receives data and control signals to and from the air supply drive portion 720, the switching portion 730, and the drive device 200 based on the control of the processor 761.

The air and water supply controller 760 is not limited to an integrated hardware device. For example, the air and water supply controller 760 may be configured by separating a part thereof into a separate hardware device and connecting the separated hardware device via a communication line. For example, the air and water supply controller 760 may be a cloud system in which the separated storage portion 763 is connected via a communication line.

The air and water supply controller 760 may further include components other than the processor 761, the memory 762, the storage portion 763, and the input-output control portion 764 shown in FIG. 15.

The air supply drive portion 720 is an air supply device that has a pump and is capable of supplying a gas such as air. The air supply drive portion 720 is connected to the atmosphere opening portion 740, the third adaptor 711, and a water supply tank 800.

The air and water supply controller 760 controls the entire air and water supply control device 700. The air and water supply controller 760 acquires the operation input received by the operation receiving portion 220 of the drive device 200. The air and water supply controller 760 controls the air supply drive portion 720 and the switching portion 730 on the basis of the acquired operation input.

That is, the air and water supply controller 760 controls the air supply drive portion 720 and the switching portion 730 on the basis of the operation input input to the operation device 300. In this embodiment, the air and water supply controller 760 controls the air supply drive portion 720 and the switching portion 730 on the basis of a touch operation and a push operation input to the first button 350.

The air and water supply controller 760 drives the air supply drive portion 720 on the basis of the operation input input to the first button 350, and causes the air supply drive portion 720 to supply air.

The air supply drive portion 720 is connected to the third adaptor 711, the atmosphere opening portion 740, and the water supply tank 800 via tubular members. The air supply drive portion 720 is capable of supplying a gas such as air to the third adaptor 711, the atmosphere opening portion 740, and the water supply tank 800 via a pipe line formed by the tubular members.

The gas supplied from the air supply drive portion 720 to the third adaptor 711 flows into the air supply pipe line 175a connected to the third adaptor 711. The gas that has flowed into the air supply pipe line 175a is sent to the air and water supply pipe line 175 of the insertion portion 110, and is ejected from the air and water supply portion 111d provided at the distal end portion 111 toward the imaging portion 111c.

The atmosphere opening portion 740 can open the pipe line that connects the air supply drive portion 720 and the atmosphere opening portion 740 to the atmosphere.

The water supply tank 800 is a container that contains a liquid such as water. The water supply tank 800 is connected to the fourth adaptor 712 via a tubular member.

When air is supplied from the air supply drive portion 720 to the water supply tank 800, a pressure inside the water supply tank 800 increases, and a liquid contained in the water supply tank 800 is sent to the fourth adaptor 712 via a pipe line that connects the water supply tank 800 and the fourth adaptor 712.

The liquid sent from the water supply tank 800 to the fourth adaptor 712 flows into the water supply pipe line 175b connected to the fourth adaptor 712. The liquid that has flowed into the water supply pipe line 175b is sent to the air and water supply pipe line 175 of the insertion portion 110, and is ejected from the air and water supply portion 111d provided at the distal end portion 111 toward the imaging portion 111c.

In this way, the air and water supply controller 760 can drive the air and water supply drive portion 720 and can eject a gas or liquid from the air and water supply portion 111d.

The switching portion 730 includes a first switching portion 730a and a second switching portion 730b.

The first switching portion 730a is a valve that can open and close the pipe line that connects the air supply drive portion 720 and the third adaptor 711 and the pipe line that connects the air supply drive portion 720 and the atmosphere opening portion 740.

The second switching portion 730b is a valve that can open and close the pipe line that connects the air supply drive portion 720 and the third adaptor 711 and the pipe line that connects the water supply tank 800 and the fourth adaptor 712.

In the pipe line that connects the air supply drive portion 720 and the third adaptor 711, the second switching portion 730b is provided so as to be able to open and close a portion downstream of a portion that the first switching portion 730a can open and close.

In the air and water supply control device 700 shown in FIG. 1 and FIG. 15, each of constituent elements provided inside a housing is connected to the third attaching and detaching portion 1503 of the endoscope 100 provided outside the housing by the air and water supply adaptor 710 (the third adaptor 711 and the fourth adaptor 712), but some of the constituent elements of the air and water supply control device 700 may be provided outside the housing.

For example, the constituent elements downstream of the air supply drive portion 720 may be provided outside the housing.

In this case, for example, a tubular member extending from the air supply drive portion 720 extends from the inside to the outside of the housing of the air and water supply control device 700.

The tubular member extending from the air supply drive portion 720 to the outside of the housing branches off outside the housing and is connected to the atmosphere opening portion 740, the water supply tank 800, and the third adaptor 711, each of which is provided outside the housing.

Additionally, outside the housing, the water supply tank 800 and the fourth adaptor 712 are connected by a tubular member, and the first switching portion 730a and the second switching portion 730b are provided in a predetermined pipe line.

In addition, the air and water supply control device 700 may not include the air and water supply controller 760, and the drive controller 260 may control the air supply drive portion 720 and the switching portion 730.

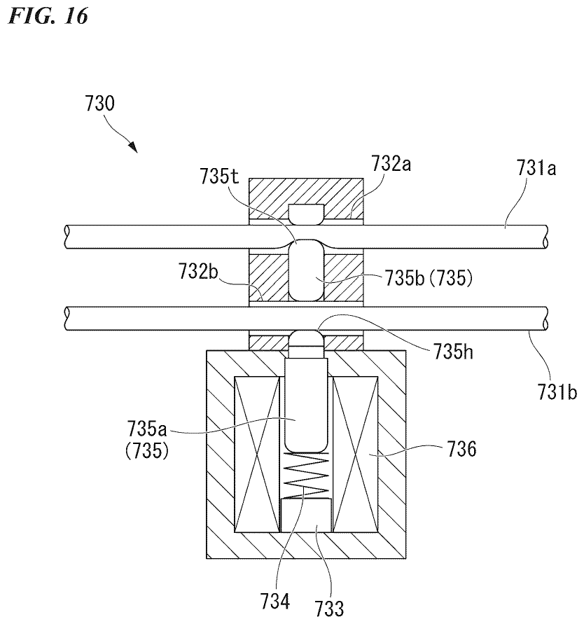

FIG. 16 is a cross-sectional view showing the switching portion 730. In this embodiment, the switching portion 730 is a dual pinch valve that can switch two pipe lines between different open and closed states.

As shown in FIG. 16, the switching portion 730 includes a fixed core 733, an elastic portion 734, a movable core 735, and a coil 736.

The fixed core 733 and the movable core 735 are connected via the elastic portion 734. The elastic portion 734 is, for example, a stretchable spring member.

The coil 736 is a cylindrical coil provided around the movable core 735. The movable core 735 moves in a direction away from the fixed core 733 by an electromagnetic force obtained by passing a current through the coil 736. At this time, the elastic portion 734 stretches in the direction in which the movable core 735 moves.

When the movable core 735 is moved in the direction away from the fixed core 733 by the electromagnetic force, and the supply of current to the coil 736 is stopped, the movable core 735 is biased by an elastic force of the elastic portion 734 and moves in a direction toward the fixed core 733. In this way, the movable core 735 moves inside the coil 736.

In the following description, the direction in which the movable core 735 moves away from the fixed core 733 is referred to as a “first direction,” and the direction in which the movable core 735 moves toward the fixed core 733 is referred to as a “second direction.”

As shown in FIG. 16, a first tube hole 732a through which a first tube 731a is inserted, and a second tube hole 732b through which a second tube 731b is inserted are provided in the switching portion 730. The first tube 731a and the second tube 731b are tubular members through whose internal spaces a gas and a liquid can pass.

An insertion hole 735h through which the second tube 731b is inserted is provided in the movable core 735. The second tube 731b is inserted through the second tube hole 732b and the insertion hole 735h.

In the cross-sectional view shown in FIG. 16, the movable core 735 has a core main body 735a connected to the elastic portion 734, and an opening and closing portion 735b provided on the first tube hole 732a side with respect to the insertion hole 735h.

The switching portion 730 shown in FIG. 16 is in a state in which the movable core 735 has been moved in the first direction by an electromagnetic force. At this time, as a distal end portion 735t of the opening and closing portion 735b enters the first tube hole 732a, the first tube 731a is pinched and crushed between the distal end portion 735t and the inner surface of the first tube hole 732a.

When the first tube 731a is crushed by the opening and closing portion 735b, the internal space of the first tube 731a becomes narrower, and the flow of the gas or liquid flowing through the internal space of the first tube 731a is blocked. That is, the first tube 731a is in a closed state, and the gas or liquid does not flow through the internal space of the first tube 731a.

At this time, the second tube 731b is inserted through the second tube hole 732b and the insertion hole 735h, and has an internal space large enough to allow the flow of the gas or liquid. That is, the second tube 731b is in an open state, and the gas or liquid can flow through the internal space of the second tube 731b.

When the supply of current to the coil 736 is stopped and the movable core 735 is moved in the second direction by the elastic portion 734, as the insertion hole 735h moves in the second direction with respect to the second tube hole 732b, the second tube 731b is pinched and crushed between the opening and closing portion 735b and the inner surface of the second tube hole 732b.

When the second tube 731b is crushed by the opening and closing portion 735b, the internal space of the second tube 731b becomes narrower, and the flow of the gas or liquid flowing through the internal space of the second tube 731b is blocked. That is, the second tube 731b is in the closed state, and the gas or liquid does not flow through the internal space of the second tube 731b.

At this time, the distal end portion 735t does not enter the first tube hole 732a. Thus, the first tube 731a has an internal space large enough to allow the flow of the gas or liquid. That is, the first tube 731a is in the open state, and the gas or liquid can flow through the internal space of the first tube 731a.

In this embodiment, when a current is supplied to the coil 736, as shown in FIG. 16, the first tube 731a is in the closed state and the second tube 731b is in the open state.

Also, when no current is supplied to the coil 736, the first tube 731a is in the open state and the second tube 731b is in the closed state.

In this way, the switching portion 730 can switch the two pipe lines between the different open and closed states. Furthermore, the air and water supply controller 760 can control the supply or stop of current to the coil 736, thereby switching the two pipe lines provided with the switching portion 730 between the different open and closed states.

As shown in FIG. 15, the first switching portion 730a is provided in a pipe line that connects the air supply drive portion 720 and the third adaptor 711 and a pipe line that connects the air supply drive portion 720 and the atmosphere opening portion 740.

In this case, for example, the pipe line that connects the air supply drive portion 720 and the third adaptor 711 is the first tube 731a shown in FIG. 16, and the pipe line that connects the air supply drive portion 720 and the atmosphere opening portion 740 is the second tube 731b shown in FIG. 16.

That is, the first switching portion 730a can switch the pipe line that connects the air supply drive portion 720 and the third adaptor 711 and the pipe line that connects the air supply drive portion 720 and the atmosphere opening portion 740 between the different open and closed states.

Further, the second switching portion 730b is provided in a pipe line that connects the air supply drive portion 720 and the third adaptor 711 and in a pipe line that connects the water supply tank 800 and the fourth adaptor 712.

In this case, for example, the pipe line that connects the air supply drive portion 720 and the third adaptor 711 is the first tube 731a shown in FIG. 16, and the pipe line that connects the water supply tank 800 and the fourth adaptor 712 is the second tube 731b shown in FIG. 16.

In other words, the second switching portion 730b can switch the pipe line that connects the air supply drive portion 720 and the third adaptor 711 and the pipe line that connects the water supply tank 800 and the fourth adaptor 712 between the different open and closed states.

### [Operation of the Endoscope System 1000]

Next, an operation of the endoscope system 1000 of this embodiment will be described.

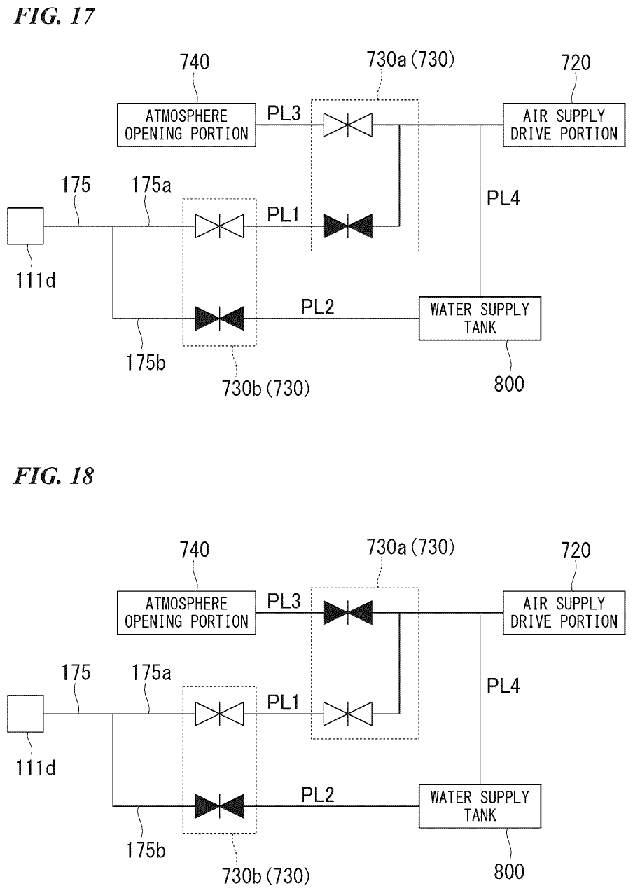

FIGS. 17, 18, 19 and 20 are schematic diagrams showing pipe lines through which a gas and a liquid pass to be sent to the air and water supply portion 111d.

The air and water supply control device 700 controls the air supply drive portion 720 and the switching portion 730 on the basis of the operation input inputted to the operation device 300, and performs a non-operation step, an air supply step, a water supply step, an atmosphere opening step, and a drainage step.

FIG. 17 is a diagram showing the non-operational step of the control method of the endoscope system 1000.

In the following description, the pipe line that extends from the air supply drive portion 720 to the air supply and water supply pipe line 175 is referred to as a “first pipe line PL1,” the pipe line that extends from the water supply tank 800 to the air supply and water supply pipe line 175 is referred to as a “second pipe line PL2,” the pipe line that branches off from the first pipe line PL1 and extends to the atmosphere opening portion 740 is referred to as a “third pipe line PL3,” and the pipe line that branches off from the first pipe line PL1 and extends to the water supply tank 800 is referred to as a “fourth pipe line PL4.”

In the endoscope 100, the gas and liquid flowing through the air and water supply pipe line 175 flows in the longitudinal direction A from the proximal end side A2 to the distal end side A1. In the first pipe line PL1, the second pipe line PL2, the third pipe line PL3, and the fourth pipe line PL4, the gas or liquid flows from the “upstream side” to the “downstream side.”

The first pipe line PL1 includes the air supply pipe line 175a. The second pipe line PL2 includes a water supply pipe line 175b.

The first pipe line PL1 and the second pipe line PL2 are connected to the upstream side (the proximal end side A2) of the air and water supply pipe line 175 in a flow direction of the gas or liquid.

Here, the term “connected” refers to a state in which the internal spaces are linked together and fluids such as a gas or liquid can pass between the internal spaces. The air supply drive portion 720 is connected to the upstream side of the first pipe line PL1. The gas supplied from the air supply drive portion 720 can flow into the internal space of the first pipe line PL1.

The water supply tank 800 is connected to the upstream side of the second pipe line PL2. The internal space of the water supply tank 800 and the internal space of the second pipe line PL2 are communicated with each other, and the liquid contained in the internal space of the water supply tank 800 can flow into the internal space of the second pipe line PL2.

The fourth pipe line PL4 branches off from the first pipe line PL1 at a portion upstream of the point from which the third pipe line PL3 branches off.

The first switching portion 730a switches the first pipe line PL1, which is located downstream in the flow direction of the gas with respect to the point from which the third pipe line PL3 branches off, and the third pipe line PL3 between the different open and closed states.

In the non-operation step shown in FIG. 17, the air and water supply control device 700 controls the first switching portion 730a to set the first pipe line PL1 to the closed state and the third pipe line PL3 to the open state.

In this embodiment, the open state refers to a state in which the pipe line is open and the gas or liquid can flow through the pipe line, and the closed state refers to a state in which the pipe line is closed and the gas or liquid cannot flow through the pipe line.

The second switching portion 730b switches the first pipe line PL1 and the fourth pipe line PL4, which are located downstream of the first switching portion 730a, between the different open and close states.

In the non-operation step shown in FIG. 17, the air and water supply control device 700 controls the second switching portion 730b to set the first pipe line PL1 to the open state and the second pipe line PL2 to the closed state.

In the non-operation step, the air and water supply control device 700 may or may not drive the air supply drive portion 720.

In the non-operation step, since the first pipe line PL1 is in the closed state due to the first switching portion 730a, and the second pipe line PL2 is in the closed state due to the second switching portion 730b, even when the air supply drive portion 720 is driven, the gas supplied from the air supply drive portion 720 and the liquid contained in the water supply tank 800 do not flow into the air and water supply pipe line 175. That is, in the non-operation step, the gas and the liquid are not ejected from the air and water supply portion 111d.

In the non-operation step, the gas supplied from the air supply drive portion 720 is sent to the atmosphere opening portion 740 through the third pipe line PL3 branching off from the first pipe line PL1.

The gas sent to the atmosphere opening portion 740 flows out, for example, into an external space of the endoscope system 1000.

FIG. 18 is a diagram showing the air supply step of the control method of the endoscope system 1000.

In the air supply step shown in FIG. 18, the air and water supply control device 700 controls the first switching portion 730a to set the first pipe line PL1 to the open state and the third pipe line PL3 to the closed state.

In the air supply step shown in FIG. 18, the air and water supply control device 700 controls the second switching portion 730b to set the first pipe line PL1 to the open state and the second pipe line PL2 to the closed state.

In the air supply step shown in FIG. 18, the air and water supply control device 700 drives the air supply drive portion 720 to cause a gas to flow from the air supply drive portion 720 into the first pipe line PL1.

In the air supply step, since the first pipe line PL1 is in the open state, and the second pipe line PL2 and the third pipe line PL3 are in the closed state, the gas supplied from the air supply drive portion 720 flows through the first pipe line PL1 into the air and water supply pipe line 175, and is ejected from the air and water supply portion 111d.

In addition, in the air supply step, since the second pipe line PL2 is in the closed state, the liquid contained in the water supply tank 800 does not flow into the air and water supply pipe line 175 and is not ejected from the air and water supply portion 111d.

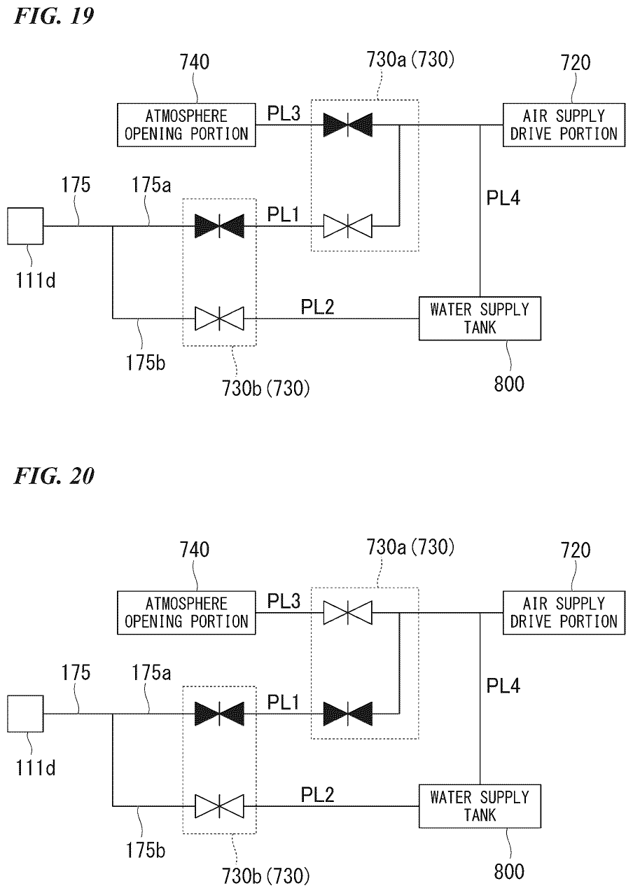

FIG. 19 is a diagram showing the water supply step of the control method of the endoscope system 1000.

In the water supply step shown in FIG. 19, the air and water supply control device 700 controls the first switching portion 730a to set the first pipe line PL1 to the open state and the third pipe line PL3 to the closed state.

In the water supply step shown in FIG. 19, the air and water supply control device 700 controls the second switching portion 730b to set the first pipe line PL1 to the closed state and the second pipe line PL2 to the open state.

In the water supply step shown in FIG. 19, the air and water supply control device 700 drives the air supply drive portion 720 to cause a gas to flow from the air supply drive portion 720 into the first pipe line PL1.

In the water supply step, since the first pipe line PL1 downstream of the point from which the fourth pipe line PL4 branches off, and the third pipe line PL3 are in the closed state, the gas supplied from the air supply drive portion 720 flows into the water supply tank 800 via the fourth pipe line PL4.

An end portion of the fourth pipe line PL4 on the downstream side is not in contact with the liquid contained in the water supply tank 800. In addition, an end portion of the second pipe line PL2 on the upstream side is in contact with the liquid contained in the water supply tank 800.

When the gas flows into the water supply tank 800 via the fourth pipe line PL4, a pressure in the water supply tank 800 increases. At this time, since the second pipe line PL2 is in the open state, the liquid contained in the water supply tank 800 is pushed out into the second pipe line PL2.

The liquid that has flowed from the water supply tank 800 into the second pipe line PL2 flows into the air and water supply pipe line 175 connected to the downstream side of the second pipe line PL2, and is ejected from the air and water supply portion 111d.

FIG. 20 is a diagram showing the atmosphere opening step of the control method of the endoscope system 1000.

In the atmosphere opening step shown in FIG. 20, the air and water supply control device 700 controls the first switching portion 730a to set the first pipe line PL1 to the closed state and the third pipe line PL3 to the open state.

In the atmosphere opening step shown in FIG. 20, the air and water supply control device 700 controls the second switching portion 730b to set the first pipe line PL1 to the closed state and the second pipe line PL2 to the open state.

In the atmosphere opening step, since the pipe line that connects the water supply tank 800 and the atmosphere opening portion 740 is in the open state, the inside of the water supply tank 800 is open to the atmosphere.

When the endoscope 100 is inserted into the body of the patient P, the air and water supply portion 111d is located inside the body of the patient P. For example, when the operator S observes the inside of the body of the patient P using the endoscope system 1000, the operator S ejects a gas such as air into the body of the patient P to make it easier to observe the inside of the body of the patient P.

At this time, the pressure inside the body of the patient P becomes higher than the atmospheric pressure. Also, a pressure inside the air and water supply pipe line 175 that communicates with the body of the patient P via the air and water supply portion 111d becomes higher than the atmospheric pressure.

When the pressure inside the body of the patient P is higher than the atmospheric pressure and the inside of the water supply tank 800 is open to the atmosphere, the liquid remaining in the second pipe line PL2 moves upstream (towards the water supply tank 800) due to a pressure difference between the inside of the body of the patient P and the inside of the water supply tank 800.

For example, when the imaging portion 111c is cleaned, the operator S performs the water supply step described above, ejects a liquid from the air and water supply portion 111d onto the imaging portion 111c, and washes away dirt adhering to the imaging portion 111c. When the liquid used for cleaning adheres to the imaging portion 111c, the liquid will obstruct the field of view of the imaging portion 111c.

Therefore, the operator S cleans the imaging portion 111c using the water supply step, and then performs the above-described air supply step to remove the liquid adhering to the imaging portion 111c by ejecting a gas from the air and water supply portion 111d onto the imaging portion 111c.

After the water supply step is performed, the liquid may remain in the air and water supply pipe line 175. By performing the air supply step after the water supply step, the liquid remaining in the air and water supply pipe line 175 is pushed out by the gas that flows into the air and water supply pipe line 175 from the first pipe line PL1, flows out of the air and water supply portion 111d, and is removed from the air and water supply pipe line 175.

In this embodiment, the operation of removing the liquid remaining in the air and water supply pipe line 175 from the air and water supply pipe line 175 by supplying air to the air and water supply pipe line 175 is referred to as the drainage step. In the drainage step, the switching portion 730 is in the same open or closed state as in the air supply step shown in FIG. 18.

When the liquid remaining in the air and water supply pipe line 175 is removed from the air and water supply pipe line 175 by the drainage step, there is a possibility that a downstream liquid level of the liquid remaining in the second pipe line PL2 will remain near a portion at which the second pipe line PL2 joins with the air and water supply pipe line 175 and the first pipe line PL1.

When the air supply step is performed in a state in which the liquid level of the liquid remaining in the second pipe line PL2 remains at the joined portion with the air and water supply pipe line 175 and the first pipe line PL1, there is a possibility that the liquid remaining in the second pipe line PL2 will be blown away by the gas flowing from the first pipe line PL1 into the air and water supply pipe line 175, and the liquid will flow into the air and water supply pipe line 175. At this time, an amount of liquid flowing into the air and water supply pipe line 175 is smaller than an amount of liquid flowing into the air and water supply pipe line 175 in the water supply step, for example.

When the liquid remaining in the second pipe line PL2 flows into the air and water supply pipe line 175 during the air supply step, there is a possibility that a water removal work for the imaging portion 111c may be hindered by the liquid flowing out of the air and water supply portion 111d. Here, the water removal work refers to a work of removing the liquid adhering to the imaging portion 111c by ejecting a gas from the air and water supply portion 111d to the imaging portion 111c.

By performing the above-described atmosphere opening step, the endoscope system 1000 can move the downstream liquid level of the liquid remaining in the second pipe line PL2 to the upstream side.

By moving the downstream liquid level of the liquid remaining in the second pipe line PL2 to the upstream side, it is possible to curb the liquid flowing into the air and water supply pipe line 175 due to the gas supplied in the air supply step, and the water removal work for the imaging portion 111c can be performed efficiently.

### [Control Method of the Endoscope System 1000]

Next, a control method of the endoscope system 1000 of this embodiment will be described.

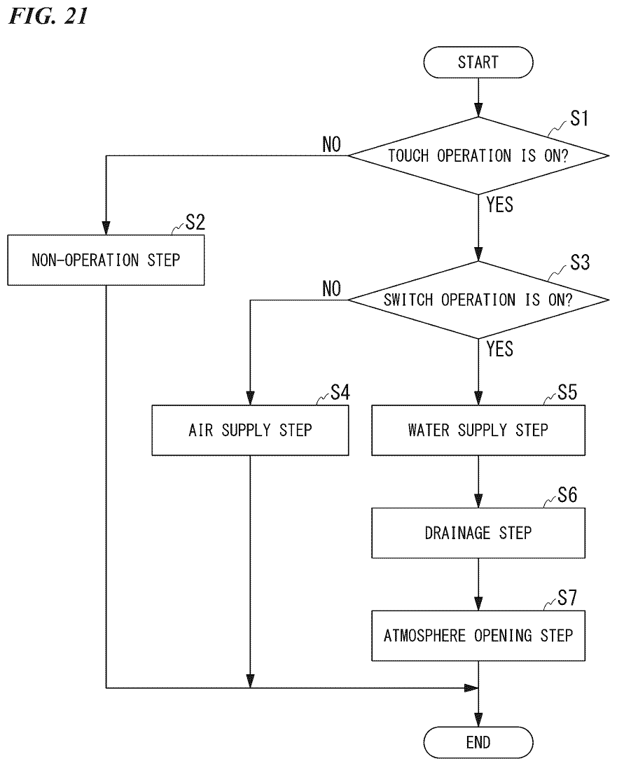

FIG. 21 is a flowchart showing the control method of the endoscope system 1000.

### (Step S1)

The control device 600 first performs Step S1 (a first operation determination step). In Step S1, the control device 600 determines whether or not a first operation (for example, a touch operation) has been input to the detection portion 350 of the operation device 300.

When the detection portion 350 does not detect the first operation, the control device 600 proceeds to Step S2.

### (Step S2)

In Step S2 (a non-operation step), the control device 600 controls the switching portion 730 to set the open and closed states of the first pipe line PL1, the second pipe line PL2, and the third pipe line PL3 to the states shown in FIG. 17.

Specifically, the air and water supply control device 700 controls the first switching portion 730a to set the first pipe line PL1 to the closed state and the third pipe line PL3 to the open state. In addition, the air and water supply control device 700 controls the second switching portion 730b to set the first pipe line PL1 to the open state and the second pipe line PL2 to the closed state.

In Step S2, the gas and liquid are not ejected from the air and water supply portion 111d. After Step S2 is performed, the control device 600 ends the control of the endoscope system 1000.

In the above-described Step S1, when the detection portion 350 detects the first operation, the control device 600 proceeds to Step S3.

### (Step S3)

In Step S3 (a second operation determination step), the control device 600 determines whether or not a second operation (for example, a switch operation or a push operation) has been input to the detection portion 350 of the operation device 300.

When the detection portion 350 does not detect the second operation, the control device 600 proceeds to Step S4.

### (Step S4)

In Step S4 (an air supply step), the control device 600 controls the switching portion 730 to set the open and closed states of the first pipe line PL1, the second pipe line PL2, and the third pipe line PL3 to the states shown in FIG. 18.

Specifically, the air and water supply control device 700 controls the first switching portion 730a to set the first pipe line PL1 to the open state and the third pipe line PL3 to the closed state. In addition, the air and water supply control device 700 controls the second switching portion 730b to set the first pipe line PL1 to the open state and the second pipe line PL2 to the closed state.

Furthermore, the air and water supply control device 700 drives the air supply drive portion 720. In Step S4, the gas supplied from the air supply drive portion 720 flows through the first pipe line PL1 into the air and water supply pipe line 175, and is ejected from the air and water supply portion 111d.

The detection portion 350 is capable of detecting an operation for performing the air supply step (the air supply operation). For example, the operator S touches the detection portion 350 and inputs a touch operation (the first operation) to the operation device 300, thereby causing the endoscope system 1000 to perform the air supply step. The operator S can make the inside of the body of the patient P easier to observe with the imaging portion 111c by having the endoscope system 1000 perform the air supply step and supplying a gas into the body of the patient P.

After Step S4 is performed, the control device 600 ends the control of the endoscope system 1000.

When the detection portion 350 detects the second operation, the control device 600 proceeds to Step S5.

### (Step S5)

In Step S5 (a water supply step), the control device 600 controls the switching portion 730 to set the open and closed states of the first pipe line PL1, the second pipe line PL2, and the third pipe line PL3 to the states shown in FIG. 19.

Specifically, the air and water supply control device 700 controls the first switching portion 730a to set the first pipe line PL1 to the open state and the third pipe line PL3 to the closed state. In addition, the air and water supply control device 700 controls the second switching portion 730b to set the first pipe line PL1 to the closed state and the second pipe line PL2 to the open state.

Furthermore, the air and water supply control device 700 drives the air supply drive portion 720. In Step S5, the gas supplied from the air supply drive portion 720 flows into the water supply tank 800 via the fourth pipe line PL4.

As the gas flows into the water supply tank 800 from the fourth pipe line PL4, the liquid contained in the water supply tank 800 flows into the second pipe line PL2 and the air and water supply pipe line 175, and the liquid is ejected from the air and water supply portion 111d.

The detection portion 350 is capable of detecting an operation for performing the water supply step (the water supply operation). For example, the operator S inputs a switch operation (the second operation) to the operation device 300 by touching the detection portion 350 and pressing the detection portion 350. The operator S causes the endoscope system 1000 to perform the water supply step by inputting the second operation to the operation device 300.

The operator S can cause the endoscope system 1000 to perform the water supply step, thereby causing the liquid to be ejected from the air and water supply portion 111d, and cleaning the imaging portion 111c with the liquid.

While the operator S is inputting the second operation to the detection portion 350, the operator S can cause the endoscope system 1000 to perform the water supply step. The operator S causes the endoscope system 1000 to perform the water supply step for an arbitrary period of time, and completes cleaning of the imaging portion 111c.

For example, when the cleaning of the imaging portion 111c is completed, the operator S releases the pressing of the detection portion 350 to stop the water supply step. The control device 600 stops the water supply step when the pressing of the detection portion 350 is released and the switch operation (the second operation) is no longer detected. That is, the detection portion 350 detects an operation for stopping the water supply step (a non-water-supply operation).

In this embodiment, the non-water-supply operation refers to an operation of releasing the pressed detection portion 350. In the case in which the first operation and the second operation are two-stage pressing operations, the non-water-supply operation refers to an operation of releasing the second-stage pressing in the detection portion 350.

When the detection portion 350 detects the non-water-supply operation, the control device 600 proceeds to Step S6.

### (Step S6)

In Step S6 (the drainage step), the control device 600 controls the switching portion 730 to set the open and closed states of the first pipe line PL1, the second pipe line PL2, and the third pipe line PL3 to the states shown in FIG. 18.

Specifically, the air and water supply control device 700 controls the first switching portion 730a to set the first pipe line PL1 to the open state and the third pipe line PL3 to the closed state. In addition, the air and water supply control device 700 controls the second switching portion 730b to set the first pipe line PL1 to the open state and the second pipe line PL2 to the closed state.

Furthermore, the air and water supply control device 700 drives the air supply drive portion 720. The operation of the air and water supply control device 700 in the drainage step is the same as the operation in the air supply step of Step S4.

In Step S6, the gas supplied from the air supply drive portion 720 flows through the first pipe line PL1 into the air and water supply pipe line 175, causing the liquid remaining in the air and water supply pipe line 175 in Step S5 to flow out of the air and water supply portion 111d.

The control device 600 can remove the liquid remaining in the air and water supply pipe line 175 by performing the drainage step.

After the water supply step in Step S5 is performed, the control device 600 automatically performs the drainage step in Step S6. The time period during which the control device 600 performs the drainage step is, for example, 0.5 seconds or less.

### (Step S7)

Next, the control device 600 performs Step S7 (an atmosphere opening step).

In Step S7, the control device 600 controls the switching portion 730 to set the open and closed states of the first pipe line PL1, the second pipe line PL2, and the third pipe line PL3 to the states shown in FIG. 20.

Specifically, the air and water supply control device 700 controls the first switching portion 730a to set the first pipe line PL1 to the closed state and the third pipe line PL3 to the open state. The air and water supply control device 700 also controls the second switching portion 730b to set the first pipe line PL1 to the closed state and the second pipe line PL2 to the open state. At this time, it is preferable that the drive of the air supply drive portion 720 is stopped.

The control device 600 reduces the pressure inside the water supply tank 800 that has increased in the water supply step of Step S5 by performing the atmosphere opening step.

The control device 600 reduces the pressure in the water supply tank 800, making the pressure in the water supply tank 800 lower than the pressure inside the body of the patient P (or inside the air and water supply pipe line 175), thereby moving the downstream liquid level of the liquid remaining in the second pipe line PL2 to the upstream side.

After the drainage step in Step S6 is performed, the control device 600 automatically performs the atmosphere opening step in Step S7. The time period during which the control device 600 performs the atmosphere opening step is, for example, 1 second or more and 1.5 seconds or less.

After Step S7 is performed, the control device 600 ends the control of the endoscope system 1000.

For example, when the control device 600 finishes the operation of Step S7, some of the liquid supplied in Step S5 is attached to the imaging portion 111c.

After the control device 600 has performed the atmosphere opening step, the operator S inputs the first operation to the operation device 300, and performs the air supply step to remove the liquid adhering to the imaging portion 111c.

At this time, the liquid level of the liquid remaining in the second pipe line PL2 is moving upstream as a result of the control device 600 performing Step S7. Therefore, the liquid remaining in the second pipe line PL2 does not flow out of the air and water supply portion 111d during the air supply step.

By performing the atmosphere opening step of Step S7 after the water supply step, the endoscope system 1000 can curb the liquid remaining in the second pipe line PL2 flowing out of the air and water supply portion 111d during the air supply step, and can efficiently perform the water removal work on the imaging portion 111c.

In the case in which the first operation is a touch operation and the second operation is a push operation, the control device 600 performs the water supply step when the detection portion 350 detects the push operation. Furthermore, the control device 600 performs the air supply step when the detection portion 350 detects a touch operation but does not detect a push operation.

The operator S can input the push operation by pressing the detection portion 350, then can release the pressing of the detection portion 350 and can maintain contact with the detection portion 350, thereby successively inputting the push operation and the touch operation.

When the first operation and the second operation are two-stage push operations (a first push operation and a second push operation), the operator S can input the first push operation and the second push operation consecutively by firmly pressing the detection portion 350 to input the second push operation, and then by reducing the force with which the detection portion 350 is pressed to transition to the first push operation.

The operator S can easily perform the cleaning operation by the water supply step and the water removal operation by the air supply step on the imaging portion 111c by successively inputting the second operation and the first operation into the operation device 300.

When the detection portion 350 detects the first operation, the control device 600 may automatically perform the drainage step of Step S6 and the atmosphere opening step of Step S7 in this order before performing the air supply step of Step S4.

Furthermore, the control device 600 may automatically perform the air supply step in Step S4 after the atmosphere opening step in Step S7. The time period during which the control device 600 automatically performs the air supply step after the atmosphere opening step is, for example, 1 second or less.

As the control device 600 automatically performs the air supply step after the atmosphere opening step, the endoscope system 1000 can automatically perform the water removal work on the imaging portion 111c.

Furthermore, after the drainage step, the control device 600 may repeat the atmosphere opening step and the air supply step in this order multiple times.

By performing the air supply step to supply a gas into the body of the patient P, the pressure inside the body of the patient P can be increased. Therefore, by repeating the atmosphere opening step and the air supply step multiple times, the atmosphere opening step can be performed with the pressure inside the body of the patient P kept high, and the liquid level of the liquid in the second pipe line PL2 can be efficiently lowered.

The endoscope system 1000 according to this embodiment includes the endoscope 100 including the air and water supply portion 111d that is provided at the distal end portion 111 of the insertion portion 110 and is capable of ejecting a gas or liquid, an air and water supply pipe line 175 that supplies the gas or liquid to the air and water supply portion 111d, and the first pipe line PL1 and the second pipe line PL2 connected to the upstream side of the air and water supply pipe line 175 in a flow direction of the gas or liquid; the air supply drive portion 720 connected to the upstream side of the first pipe line PL1 and capable of supplying the gas to the first pipe line PL1; the water supply tank 800 that is connected to the upstream side of the second pipe line PL2 and contains the liquid; the atmosphere opening portion 740 connected to the third pipe line PL3 branching off from the first pipe line PL1 and capable of opening the first pipe line PL1 to the atmosphere; a switching portion 730 capable of opening and closing the first pipe line PL1 which is located downstream of the point from which the third pipe line PL3 branches off, the second pipe line PL2, and the third pipe line PL3; and a control device 600 that controls the air supply drive portion 720 and the switching portion 730.

The water supply tank 800 is connected to a portion of the first pipe line PL1 which is located upstream of the switching portion 730.

The control device 600 performs a water supply step of setting the first pipe line PL1 to the closed state, the second pipe line PL2 to the open state, and the third pipe line PL3 to the closed state, and driving the air supply drive portion 720 to eject a liquid from the air and water supply portion 111d, an air supply step of setting the first pipe line PL1 to the open state, the second pipe line PL2 to the closed state, and the third pipe line PL3 to the closed state, and driving the air supply drive portion 720 to eject a gas from the air and water supply portion 111d, and the atmosphere opening step of setting the first pipe line PL1 to the closed state, the second pipe line PL2 to the open state, and the third pipe line PL3 to the open state to reduce the pressure in the water supply tank 800.

Even when the liquid remains in the air and water supply pipe line 175, the endoscope system 1000 can perform an operation similar to the air supply step to send a gas to the air and water supply pipe line 175, thereby causing the liquid remaining in the air and water supply pipe line 175 to flow out and be removed from the air and water supply portion 111d.

Furthermore, even when the liquid remains in the second pipe line PL2, the endoscope system 1000 can move the liquid level of the liquid remaining in the second pipe line PL2 upstream by performing the atmosphere opening step.

As a result, the endoscope system 1000, the control device 600, and the control method for the endoscope system 1000 can be provided that can curb the liquid being ejected along with the gas when the gas is ejected from the air and water supply portion 111d which is capable of ejecting both the liquid and gas.

Although one embodiment of the present disclosure has been described in detail above with reference to the drawings, the specific configuration is not limited to this embodiment, and design changes and the like that do not deviate from the gist of the present disclosure are also included. Furthermore, the constituent elements shown in the above-described embodiment and modified examples can be appropriately combined to form a configuration.

### Modified Example 1

In the above embodiment, the operator S operates the endoscope 100 with his/her right hand R and while operating the operation device 300 with his/her left hand L. However, the manner of use of the endoscope system 1000 is not limited thereto. The operator S may operate the operation device 300 with his/her right hand R while operating the endoscope 100 with his/her left hand L. In this case, the operation device 300 is optimized for easy operation with the right hand R.

### Modified Example 2

In the above embodiment, the endoscope system 1000 may further include a known endoscope mounting mechanism such as Smart Shooter (registered trademark). By using the endoscope mounting mechanism, the operator S can perform an advancement and retreatment operation of the treatment tool 400 while holding the insertion portion 110 with the right hand R.

### Modified Example 3

In the above embodiment, the endoscope system 1000 is an electric endoscope system in which the bending portion 112 is bent by the drive device 200 on the basis of an operation input to the operation device 300, but it may also be a manual endoscope in which the bending operation is performed manually without using the drive device 200.

### Modified Example 4

In the above embodiment, the switching portion 730 is a dual pinch valve capable of switching two pipe lines between different open and closed states, but the aspect of the switching portion is not limited thereto.

The switching portion may be a valve provided in each of the first pipe line PL1, the second pipe line PL2, and the third pipe line PL3, and capable of independently opening and closing each of the first pipe line PL1, the second pipe line PL2, and the third pipe line PL3.

The programs in each of the embodiments may be recorded on a computer-readable recording medium, and the programs recorded on the recording medium may be read into a computer system and executed to realize the present disclosure. The term “computer system” includes hardware such as an OS and peripheral devices. In addition, the term “computer-readable recording medium” refers to portable media such as flexible disks, optical magnetic disks, ROMs, and CD-ROMs, as well as storage devices such as hard disks built into computer systems. Furthermore, the term “computer-readable recording medium” may also include something that dynamically holds a program for a short period of time, such as a communication line when a program is transmitted via a network such as the Internet or a communication line such as a telephone line, or something that holds a program for a certain period of time, such as volatile memory inside a computer system that is a server or client in that case. Furthermore, the above program may be for realizing some of the functions described above, and may also be capable of realizing the functions described above in combination with a program already recorded in the computer system.

### (1st Aspect)

An endoscope system comprising:
an endoscope including an air and water supply portion provided at a distal end portion of an insertion portion and configured to eject a gas or liquid, an air and water supply pipe line configured to supply the gas or liquid to the air and water supply portion, and a first pipe line and a second pipe line which are connected to an upstream side of the air and water supply pipe line in a flow direction of the gas or liquid;
an air supply drive portion connected to the upstream side of the first pipe line and configured to supply the gas to the first pipe line;
a water supply tank connected to the upstream side of the second pipe line and configured to contain the liquid;
an atmosphere opening portion connected to a third pipe line branching off from the first pipe line and configured to open the first pipe line to an atmosphere;
a switching portion configured to open and close each of the first pipe line which is located downstream of a point from which the third pipe line branches off, the second pipe line, and the third pipe line; and
a control device configured to control the air supply drive portion and the switching portion,
wherein the water supply tank is connected to a portion of the first pipe line on the upstream side of the switching portion, and
the control device performs a water supply step of setting the first pipe line to a closed state, the second pipe line to an open state, and the third pipe line to a closed state, and driving the air supply drive portion to cause the liquid to be ejected from the air and water supply portion, an air supply step of setting the first pipe line to the open state, the second pipe line to the closed state, and the third pipe line to the closed state, and driving the air supply drive portion to eject the gas from the air and water supply portion, and an atmosphere opening step of setting the first pipe line to the closed state, the second pipe line to the open state, and the third pipe line to the open state to reduce a pressure in the water supply tank.

### (2nd Aspect)

The endoscope system according to the 1st aspect, wherein the control device performs the atmosphere opening step to make the pressure in the water supply tank lower than a pressure in the air and water supply pipe line, and thus moves a liquid level of the liquid remaining in the second pipe line on the downstream side to the upstream side.

### (3rd Aspect)

The endoscope system according to the 2nd aspect, wherein the control device performs a drainage step in which the liquid remaining in the air and water supply pipe line is ejected from the air and water supply portion by supplying the gas to the air and water supply pipe line, and the atmosphere opening step in this order after the water supply step or before the air supply step.

### (4th Aspect)

The endoscope system according to the 3rd aspect, wherein the control device repeats the atmosphere opening step and the air supply step in this order multiple times after the drainage step.

### (5th Aspect)

The endoscope system according to the 3rd aspect, further comprising a detection portion communicatively connected to the control device and configured to detect a water supply operation for performing the water supply step and a non-water-supply operation for stopping the water supply step,
wherein the control device performs the water supply step when the detection portion detects the water supply operation, stops the water supply step when the detection portion detects the non-water-supply operation, and performs the drainage step, the atmosphere opening step, and the air supply step in this order.

### (6th Aspect)

The endoscope system according to the 3rd aspect, further comprising a detection portion communicatively connected to the control device and configured to detect an air supply operation for performing the air supply step,
wherein the control device performs the drainage step, the atmosphere opening step, and the air supply step in this order when the detection portion detects the air supply operation.

### (7th Aspect)

The endoscope system according to the 5th or 6th aspect, wherein the detection portion is configured to detect a first operation and a second operation that is different from the first operation, and
the control device performs the air supply step when the detection portion detects the first operation, and performs the water supply step when the detection portion detects the second operation.

### (8th Aspect)

The endoscope system according to any one of the 1st to 6th aspect, wherein the switching portion includes a first switching portion configured to switch the first pipe line and the third pipe line to different open and closed states, and a second switching portion provided downstream of the first switching portion and configured to switch the first pipe line and the second pipe line to the different open and closed states.

### (9th Aspect)

A control device which controls an endoscope system including an air and water supply portion, an air and water supply pipe line configured to supply a gas or liquid to the air and water supply portion, a first pipe line and a second pipe line connected to the air and water supply pipe line, an air supply drive portion configured to supply the gas to the first pipe line, a water supply tank connected to the first pipe line and the second pipe line and configured to contain the liquid, an atmosphere opening portion connected to a third pipe line branching off from the first pipe line, and a switching portion configured to open and close each of the first pipe line which is located downstream in a gas flow direction with respect to a point from which the third pipe branches off, the second pipe line, and the third pipe line, the control device performing:
a water supply step of controlling the switching portion to set the first pipe line to a closed state, the second pipe line to an open state, and the third pipe line to the closed state, and driving the air supply drive portion to eject the liquid from the air and water supply portion;
an air supply step of setting the first pipe line to the open state, the second pipe line to the closed state, and the third pipe line to the closed state, and driving the air supply drive portion to eject the gas from the air and water supply portion; and
an atmosphere opening step of setting the first pipe line to the closed state, the second pipe line to the open state, and the third pipe line to the open state to reduce a pressure in the water supply tank.

### (10th Aspect)

The control device according to the 9th aspect, wherein a liquid level of the liquid remaining in the second pipe line on a downstream side in the flow direction is moved to an upstream side in the flow direction by performing the atmosphere opening step to make a pressure in the water supply tank lower than a pressure in the air and water supply pipe line.

### (11th Aspect)

The control device according to the 10th aspect, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

### (12th Aspect)

The control device according to the 11th aspect, wherein after the drainage step, the atmosphere opening step and the air supply step are repeated in this order multiple times.

### (13th Aspect)

The control device according to the 11th or 12th aspect, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.

### (14th Aspect)

The control device according to the 11th aspect or 12th aspect, wherein when a detection portion configured to detect an air supply operation for performing the air supply step detects the air supply operation, the drainage step, the atmosphere opening step, and the air supply step are performed in this order.

### (15th Aspect)

A control method for an endoscope system which includes an air and water supply portion, an air and water supply pipe line configured to supply a gas or liquid to the air and water supply portion, a first pipe line and a second pipe line connected to the air and water supply pipe line, an air supply drive portion configured to supply the gas to the first pipe line, a water supply tank connected to the first pipe line and the second pipe line and configured to contain the liquid, an atmosphere opening portion connected to a third pipe line branching off from the first pipe line, and a switching portion configured to open and close each of the first pipe line which is located downstream in a gas flow direction with respect to a point from which the third pipe line branches off, the second pipe line, and the third pipe line, the control method comprising:
a water supply step of controlling the switching portion to set the first pipe line to a closed state, the second pipe line to an open state, and the third pipe line to the closed state, and driving the air supply drive portion to eject the liquid from the air and water supply portion;
an air supply step of setting the first pipe line to the open state, the second pipe line to the closed state, and the third pipe line to the closed state, and driving the air supply drive portion to eject the gas from the air and water supply portion; and
an atmosphere opening step of setting the first pipe line to the closed state, the second pipe line to the open state, and the third pipe line to the open state to reduce a pressure in the water supply tank.

### (16th Aspect)

The control method for an endoscope system according to the 15th aspect, wherein a liquid level of the liquid remaining in the second pipe line on a downstream side in the flow direction is moved to an upstream side in the flow direction by performing the atmosphere opening step to make a pressure in the water supply tank lower than a pressure in the air and water supply pipe line.

### (17th Aspect)

The control method for an endoscope system according to the 16th aspect, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

### (18th Aspect)

The control method for an endoscope system according to the 17th aspect, wherein after the drainage step, the atmosphere opening step and the air supply step are repeated in this order multiple times.

### (19th Aspect)

The control method for an endoscope system according to the 17th or 18th aspect, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.

### (20th Aspect)

The control method for an endoscope system according to the 17th or 18th aspect, wherein when a detection portion configured to detect an air supply operation for performing the air supply step detects the air supply operation, the drainage step, the atmosphere opening step, and the air supply step are performed in this order.

### (1 st Aspect)

A control device for use with an endoscope, configured to:
set a first pipe line to a closed state, a second pipe line to an open state, and a third pipe line to a closed state, to cause liquid to be ejected from an air and water supply portion;
set the first pipe line to an open state, the second pipe line to a closed state, and the third pipe line to the closed state, to cause gas to be ejected from the air and water supply portion; and
set the first pipe line to the closed state, the second pipe line to the open state, and the third pipe line to an open state, to reduce a pressure in a water supply tank.

### (2nd Aspect)

An endoscope system comprising:
the control device according to 1st aspect;
an air and water supply portion;
a supply pipe line configured to supply gas or liquid to the air and water supply portion;
the first pipe line and the second pipe line which are connected to the supply pipe line;
an air supply drive portion connected to the first pipe line and configured to supply gas;
a water supply tank connected to the second pipe line; and
the third pipe line branching off from the first pipe line and configured to open the first pipe line to an atmosphere.

### (3rd Aspect)

The endoscope system according to 2nd aspect, wherein the control device makes the pressure in the water supply tank lower than a pressure in the air and water supply pipe line, and thus moves a liquid level of the liquid remaining in the second pipe line on a downstream side to an upstream side.

### (4th Aspect)

The endoscope system according to 3rd aspect, wherein the control device performs a drainage step in which the liquid remaining in the air and water supply pipe line is ejected from the air and water supply portion by supplying the gas to the air and water supply pipe line, and the atmosphere opening step in this order after the water supply step or before the air supply step.

### (5th Aspect)

The endoscope system according to 4th aspect, wherein the control device repeats the atmosphere opening step and the air supply step in this order multiple times after the drainage step.

### (6th Aspect)

The endoscope system according to 4th aspect, further comprising a detection portion communicatively connected to the control device and configured to detect a water supply operation for performing the water supply step and a non-water-supply operation for stopping the water supply step,
wherein the control device performs the water supply step when the detection portion detects the water supply operation, stops the water supply step when the detection portion detects the non-water-supply operation, and performs the drainage step, the atmosphere opening step, and the air supply step in this order.

### (7th Aspect)

The endoscope system according to 4th aspect, further comprising a detection portion communicatively connected to the control device and configured to detect an air supply operation for performing the air supply step,
wherein the control device performs the drainage step, the atmosphere opening step, and the air supply step in this order when the detection portion detects the air supply operation.

### (8th Aspect)

The endoscope system according to 6th aspect, wherein the detection portion is configured to detect a first operation and a second operation that is different from the first operation, and
the control device performs the air supply step when the detection portion detects the first operation, and performs the water supply step when the detection portion detects the second operation.

### (9th Aspect)

The endoscope system according to 2nd aspect, wherein the switching portion includes a first switching portion configured to switch the first pipe line and the third pipe line to different open and closed states, and a second switching portion provided downstream of the first switching portion and configured to switch the first pipe line and the second pipe line to the different open and closed states.

### (10th Aspect)

A control device which controls an endoscope system including an air and water supply portion, an air and water supply pipe line configured to supply a gas or liquid to the air and water supply portion, a first pipe line and a second pipe line connected to the air and water supply pipe line, an air supply drive portion configured to supply the gas to the first pipe line, a water supply tank connected to the first pipe line and the second pipe line and configured to contain the liquid, an atmosphere opening portion connected to a third pipe line branching off from the first pipe line, and a switching portion configured to open and close each of the first pipe line which is located downstream in a gas flow direction with respect to a point from which the third pipe branches off, the second pipe line, and the third pipe line, the control device performing:
a water supply step of controlling the switching portion to set the first pipe line to a closed state, the second pipe line to an open state, and the third pipe line to the closed state, and driving the air supply drive portion to eject the liquid from the air and water supply portion;
an air supply step of setting the first pipe line to the open state, the second pipe line to the closed state, and the third pipe line to the closed state, and driving the air supply drive portion to eject the gas from the air and water supply portion; and
an atmosphere opening step of setting the first pipe line to the closed state, the second pipe line to the open state, and the third pipe line to the open state to reduce a pressure in the water supply tank.

### (11th Aspect)

The control device according to 10th aspect, wherein a liquid level of the liquid remaining in the second pipe line on a downstream side in the flow direction is moved to an upstream side in the flow direction by performing the atmosphere opening step to make a pressure in the water supply tank lower than a pressure in the air and water supply pipe line.

### (12th Aspect)

The control device according to 11th aspect, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

### (13th Aspect)

The control device according to 12th aspect, wherein after the drainage step, the atmosphere opening step and the air supply step are repeated in this order multiple times.

### (14th Aspect)

The control device according to 10th aspect, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.

### (15th Aspect)

The control device according to 10th aspect, wherein when a detection portion configured to detect an air supply operation for performing the air supply step detects the air supply operation, the drainage step, the atmosphere opening step, and the air supply step are performed in this order.

### (16th Aspect)

A control method for an endoscope system which includes an air and water supply portion, an air and water supply pipe line configured to supply a gas or liquid to the air and water supply portion, a first pipe line and a second pipe line connected to the air and water supply pipe line, an air supply drive portion configured to supply the gas to the first pipe line, a water supply tank connected to the first pipe line and the second pipe line and configured to contain the liquid, an atmosphere opening portion connected to a third pipe line branching off from the first pipe line, and a switching portion configured to open and close each of the first pipe line which is located downstream in a gas flow direction with respect to a point from which the third pipe line branches off, the second pipe line, and the third pipe line, the control method comprising:
a water supply step of controlling the switching portion to set the first pipe line to a closed state, the second pipe line to an open state, and the third pipe line to the closed state, and driving the air supply drive portion to eject the liquid from the air and water supply portion;
an air supply step of setting the first pipe line to the open state, the second pipe line to the closed state, and the third pipe line to the closed state, and driving the air supply drive portion to eject the gas from the air and water supply portion; and
an atmosphere opening step of setting the first pipe line to the closed state, the second pipe line to the open state, and the third pipe line to the open state to reduce a pressure in the water supply tank.

### (17th Aspect)

The control method for an endoscope system according to 16th aspect, wherein a liquid level of the liquid remaining in the second pipe line on a downstream side in the flow direction is moved to an upstream side in the flow direction by performing the atmosphere opening step to make a pressure in the water supply tank lower than a pressure in the air and water supply pipe line.

### (18th Aspect)

The control method for an endoscope system according to 17th aspect, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

### (19th Aspect)

The control method for an endoscope system according to 18th aspect, wherein after the drainage step, the atmosphere opening step and the air supply step are repeated in this order multiple times.

### (20th Aspect)

The control method for an endoscope system according to 18th aspect, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.

What is claimed is:

1. A control device for use with an endoscope, configured to:
set a first pipe line to a closed state, a second pipe line to an open state, and a third pipe line to a closed state, to cause liquid to be ejected from an air and water supply portion;
set the first pipe line to an open state, the second pipe line to a closed state, and the third pipe line to the closed state, to cause gas to be ejected from the air and water supply portion; and
set the first pipe line to the closed state, the second pipe line to the open state, and the third pipe line to an open state, to reduce a pressure in a water supply tank.

2. An endoscope system comprising:
the control device according to claim 1;
an air and water supply portion;
a supply pipe line configured to supply gas or liquid to the air and water supply portion;
the first pipe line and the second pipe line which are connected to the supply pipe line;
an air supply drive portion connected to the first pipe line and configured to supply gas;
a water supply tank connected to the second pipe line; and
the third pipe line branching off from the first pipe line and configured to open the first pipe line to an atmosphere.

3. The control device according to claim 1, wherein the control device is configured to make the pressure in the water supply tank lower than a pressure in the air and water supply pipe line, and thus moves a liquid level of the liquid remaining in the second pipe line on a downstream side to an upstream side.

4. The control device according to claim 3, wherein, at a timing after the water supply step or before the air supply step, the control device is configured to, in this order:
perform a drainage step in which the liquid remaining in the air and water supply pipe line is ejected from the air and water supply portion by supplying the gas to the air and water supply pipe line; and
perform the atmosphere opening step.

5. The control device according to claim 4, wherein the control device is configured to repeat the atmosphere opening step and the air supply step in this order multiple times after the drainage step.

6. The control device according to claim 4, further comprising a detection portion communicatively connected to the control device and configured to detect a water supply operation for performing the water supply step and a non-water-supply operation for stopping the water supply step,
wherein the control device is configured to, in this order:
perform the water supply step when the detection portion detects the water supply operation;
stop the water supply step when the detection portion detects the non-water-supply operation; and
perform the drainage step, the atmosphere opening step, and the air supply step.

7. The control device according to claim 4, further comprising a detection portion communicatively connected to the control device and configured to detect an air supply operation for performing the air supply step,
wherein, when the detection portion detects the air supply operation, the control device is configured to, in this order:
perform the drainage step;
perform the atmosphere opening step; and
perform the air supply step.

8. The control device according to claim 6, wherein the detection portion is configured to detect a first operation and a second operation that is different from the first operation, and
the control device is configured to:
perform the air supply step when the detection portion detects the first operation; and
perform the water supply step when the detection portion detects the second operation.

9. The endoscope system according to claim 2, wherein the switching portion includes:
a first switching portion configured to switch the first pipe line and the third pipe line to different open and closed states; and
a second switching portion provided downstream of the first switching portion and configured to switch the first pipe line and the second pipe line to the different open and closed states.

10. A control device which controls an endoscope system, the control device performing:
a water supply step of controlling a switching portion to set a first pipe line to a closed state, a second pipe line to an open state, and a third pipe line to a closed state, and driving an air supply drive portion to eject a liquid from an air and water supply portion;
an air supply step of setting the first pipe line to an open state, the second pipe line to a closed state, and the third pipe line to a closed state, and driving the air supply drive portion to eject a gas from the air and water supply portion; and
an atmosphere opening step of setting the first pipe line to the closed state, the second pipe line to the open state, and the third pipe line to the open state to reduce a pressure in a water supply tank.

11. The control device according to claim 10, wherein a liquid level of the liquid remaining in the second pipe line on a downstream side in a flow direction is moved to an upstream side in the flow direction by performing the atmosphere opening step to make a pressure in the water supply tank lower than a pressure in an air and water supply pipe line configured to supply the gas or liquid to the air and water supply portion.

12. The control device according to claim 11, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

13. The control device according to claim 12, wherein after the drainage step, the atmosphere opening step and the air supply step are repeated in this order multiple times.

14. The control device according to claim 10, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.

15. The control device according to claim 10, wherein when a detection portion configured to detect an air supply operation for performing the air supply step detects the air supply operation, the drainage step, the atmosphere opening step, and the air supply step are performed in this order.

16. A control method for an endoscope system, the control method comprising:
a water supply step of controlling a switching portion to set a first pipe line to a closed state, a second pipe line to an open state, and a third pipe line to a closed state, and driving an air supply drive portion to eject a liquid from an air and water supply portion;
an air supply step of setting the first pipe line to an open state, the second pipe line to a closed state, and the third pipe line to a closed state, and driving the air supply drive portion to eject a gas from the air and water supply portion; and
an atmosphere opening step of setting the first pipe line to the closed state, the second pipe line to the open state, and the third pipe line to an open state to reduce a pressure in a water supply tank.

17. The control method for an endoscope system according to claim 16, wherein a liquid level of the liquid remaining in the second pipe line on a downstream side in a flow direction is moved to an upstream side in the flow direction by performing the atmosphere opening step to make a pressure in the water supply tank lower than a pressure in an air and water supply pipe line configured to supply the gas or liquid to the air and water supply portion.

18. The control method for an endoscope system according to claim 17, wherein after the water supply step or before the air supply step, a drainage step of supplying the gas to the air and water supply pipe line to eject the liquid remaining in the air and water supply pipe line from the air and water supply portion, and the atmosphere opening step are performed in this order.

19. The control method for an endoscope system according to claim 18, wherein after the drainage step, the atmosphere opening step and the air supply step are repeated in this order multiple times.

20. The control method for an endoscope system according to claim 18, wherein when a detection portion configured to detect a water supply operation for performing the water supply step and a non-water-supply operation for stopping the water supply step detects the water supply operation, the water supply step is performed, and
when the detection portion detects the non-water-supply operation, the water supply step is stopped, and the drainage step, the atmosphere opening step, and the air supply step are performed in this order.
